(19) United States
(12) Patent Application Publication
(10) Pub. No.: US 2004/0132675 A1
(43) Pub. Date: Jul. 8, 2004

(54) Method for treating cancer and increasing hematocrit levels

(76) Inventors: Calvin Kuo, Palo Alto, CA (US); Richard Mulligan, Cambridge, MA (US)

Correspondence Address: David S Resnick, Nixon Peabody, 101 Federal Street, Boston, MA, 02110, US

(21) Appl. No.: 10/467,509

(22) PCT Filed: Feb. 8, 2002
(86) PCT No.: PCT/US02/03531

(51) Int. Cl. A61K 48/00

(52) U.S. Cl. 514/44; 424/93.2

(57) ABSTRACT

The present invention provides a method for inhibiting undesired angiogenesis including tumor-associated angiogenesis. The invention further provides a method for increasing the number of red blood cells or hematocrit in the circulation in subjects in need thereof. The invention also provides a method for simultaneously treating low hematocrit and undesired angiogenesis. Additionally, the present inventio provides a method for determining efficacy or endpoint of treatment with one or more VEGF-inhibitors.

### BACKGROUND OF THE INVENTION

1. Field of the Invention

The present invention relates to methods for treatment of cancer and low hematocrit levels. Specifically, the invention relates to methods of treating large preexisting tumors. Additionally the invention relates to methods of treating low hematocrit levels. The invention further relates to methods of determining the efficacy of VEGF-inhibitor related treatments.

2. Technical Background

Angiogenesis, the growth of new blood vessels from existing endothelium is tightly controlled by opposing effects of positive and negative regulators. At least three families of receptor tyrosine kinases have been implicated in positive angiogenic regulation, the VEGF receptors (Flk1, Flt1), the TIE receptors (TIE1, TIE2), and the ephB4/ephrin B2 system [Ferrara et al., Nat Med 5:1359-1364, 1999; Gale et al., Genes Dev 13:1055-66, 1999]. On the other hand, putative negative angiogenic regulators, such as angiostatin and endostatin, have recently been identified [O'Reilly et al., Cell 88:277-85, 1997; O'Reilly et al., Cell 79:315-28, 1994].

Under certain pathological conditions, including proliferative retinopathies, rheumatoid arthritis, psoriasis and cancer, positive regulators prevail and angiogenesis contributes to disease progression [reviewed in Folkman, Nat Med 1:27-31, 1995]. For example, the quantity of blood vessels in tumor tissue is a strong negative prognostic indicator in breast and prostate cancer, brain tumors and melanoma [Weidner et al., J Natl Cancer Inst 84:1875-1887, 1992; Weidner et al., Am J Pathol 143:401-409, 1993; Li et al., Lancet 344:82-86, 1994; Foss et al., Cancer Res 56:2900-2903, 1996].

Vascular endothelial growth factor (VEGF) plays a key role as a positive regulator of physiological and pathological angiogenesis. Although produced by a number of different cells, VEGF appears to act selectively on endothelial cells, stimulating angiogenesis both in vitro and in vivo. VEGF is directly involved in promotion of endothelial cell permeability, growth and migration, and it also serves as a survival factor for newly formed blood vessels. In addition, VEGF stimulates the expression of tissue plasminogen activator, urokinase plasminogen activator, collagenases and matrix metalloproteinases, which are involved in the degradation of the extracellular matrix needed for endothelial cell migration [Pepper et al., Cell Differ Dev 32:319-27, 1990; Rifkin et al., Cell Differ Dev 32:313-318, 1990; Tolnay et al., J Cancer Res Clin Oncol 124:291-296, 1998; Zucker et al., Int J Cancer 75:780-786, 1998].

VEGF (VEGF-A) is a member of a growing family of growth factors comprising of at least six different proteins: PIGF, VEGF-B, VEGF-C, VEGF-D, and orf virus VEGF (VEGF-E). VEGF occurs as different isoforms encoded by splice variants of a single gene containing 8 exons. In the human, at least five different isoforms exist: VEGF₁₂₁, VEGF₁₄₃, VEGF₁₆₅,VEGF₁₈₉ and VEGF₂₀₆ [Veikkola et al., Semin Cancer Biol 9: 211-20, 1999]. Although the different isoforms exhibit identical biological activity, they differ in their binding to heparin and to the extracellular matrix.

The VEGFs mediate angiogenic signals to the vascular endothelium via high affinity receptor tyrosine kinases, designated VEGFR-1 (Flt1), VEGFR-2 (Flk1/KDR, Flk1 is the mouse homolog of human KDR) and VEGFR-3 (Flt4), characterized by seven immunoglobulin-like domains in the extracellular region, a single transmembrane domain, and an intracellular split tyrosine-kinase domain [Id.]. These VEGFRs bind distinct subsets of VEGF family members. For example, Flt1 binds PIGF, VEGF-A and VEGF-B, while Flk1 binds VEGF-A, -C and -D. VEGF-A has been suggested to bind to VEGFR-1/Flt1 on cell membranes with higher affinity than VEGFR-2/Flk1/KDR [Waltenberger et al., J Biol Chem 269:26988-95, 1994], although this differential affinity may be less pronounced with truncated soluble versions of Flk1 and Flt1 [Keyt et al., J Biol Chem 271:5638-46, 1996].

The importance of VEGF mediated processes in angiogenesis has been shown in knock-out studies: mice lacking a single allele for the VEGF gene, both alleles of the Flt1 or Flk1 genes are unable to survive beyond embryonal stages because of distinct abnormalities in vessel formation [Carmeliet et al., Nature 380:435-439, 1996; Ferrara et al., Nature 380:439-442, 1996; Shalaby et al., Nature 376:62-66, 1995]

Several molecules have been identified to regulate the expression of VEGF. For example, the expression of VEGF is highly regulated by hypoxia mediated by a family of hypoxia-inducible transcription factors (HIF), providing a physiologic feedback mechanism to accommodate insufficient tissue oxygenation by promoting blood vessel formation [Carmeliet et al., Ann NY Acad Sci 902:249-62, 2000]. In both breast and prostate cancer VEGF levels are augmented by the presence of sex hormones [Joseph et al., Cancer Res 57:1054-4, 1997; Scott et al., Int J Cancer 75:706-12, 1998]. Cytokines, such as epidermal growth factor (EGF) and transforming growth factor beta (TGF-β), may also stimulate the expression of VEGF [Takahashi et al., Int J Cancer 79:34-8, 1998]. Both VEGF mRNA and protein are markedly upregulated in the vast majority of human tumors and VEGF overexpression in cancer patients is associated with poor prognosis and low survival [Paley et al., Cancer 80:98-106, 1997]. In tumors, VEGF is not produced by endothelial cells, but instead by tumor cells or tumor stroma, consistent with a paracrine mode of action [Ferrara et al., Nat Med 5:1359-1364, 1999; Fukumura et al., Cancer Res 59:99-106, 1998].

The action of VEGF and its receptors on endothelial cells is a strong permissive factor for tumor growth, and VEGF inhibitors are prototypical antiangiogenic cancer therapeutics which target the tumor vasculature. For example, the growth of human tumor xenografts in nude mice could be inhibited by neutralizing antibodies to VEGF or expression of antisense sequence to VEGF mRNA, by the expression of dominant-negative VEGF receptor Flk1 or by low molecular weight inhibitors of Flk1 tyrosine kinase activity [Kim et al., Nature 362:841-844, 1993; Saleh et al., Cancer Res 56:393-401, 1996; Millauer et al., Cancer Res 56:1615-1620, 1996; Millauer et al., Nature 367:576-579, 1994; Strawn et al., Cancer Res 56:3540-3545, 1996] The incidence tumor metastases was also found to be reduced by VEGF antagonists [Claffey et al., Cancer Res 56:172-181, 1996]

Recently, the administration of several tumor-derived circulating proteins have also been proposed as an alternative non-VEGF dependent strategy for systemic inhibition of angiogenesis. In particular, both human and murine forms of angiostatin, a proteolytic fragment of plasminogen, have been described to exert potent anti-angiogenic and anti-tumor activities in a variety of murine tumor models, extending to frank regression of tumors [O'Reilly et al., Cell 79:315-28, 1994; O'Reilly et al., Nat Med2:689-92, 1996]. Similarly, a C-terminal fragment of collagen XVIII, termed endostatin, has been reported to exhibit anti-angiogenic and tumor-regressing activities accompanied by a lack of acquired tumor resistance [O'Reilly et al., Cell 88:277-85, 1997; Boehm et al., Nature 390:404-7, 1997]

Gene therapy approaches for delivery of anti-angiogenic factors have several advantages over conventional administration, including chronic production, lack of peak-and-trough pharmacokinetics, and potential economics of production of vectors versus protein. Although several previous reports have documented the anti-tumor effects of vector-mediated delivery of angiostatin, endostatin, soluble Flt1 ectodomains, and soluble neuropilin (sNRP) domains, [Takayama et al., Cancer Res 60:2169-77, 2000; Griscelli et al., Proc Natl Acad Sci USA 95:6367-6372, 1998; Blezinger et al., Nat Biotechnol 17:343-8 1999; Chen et al., Cancer Res 59:3308-3312, 1999; Sauter et al., Proc Natl Acad Sci USA 97:4802-4807, 2000; Feldman et al., Cancer Res 60:1503-1506, 2000], such gene therapy approaches have not been shown to potently inhibit large (>100 mm³) aggressive pre-existing tumors by systemic delivery. For example, while it has been shown that tumor lines stably transfected with angiostatin cDNA exhibit impaired tumor growth, systemic gene therapy with angiostatin has not been shown to strongly suppress pre-existing tumor growth [Griscelli et al., Proc Natl Acad Sci USA 95:6367-6372, 1998; Chen et al., Cancer Res 59:3308-3312, 1999]., Similarly, while several studies report the inhibition of tumor growth and metastases in mice after vector-mediated delivery of endostatin, no strong activity against pre-existing tumors has been reported [Blezinger et al., Nat Biotechnol 17:343-348 1999; Chen et al., Cancer Res 59:3308-3312, 1999; Sauter et al., Proc Natl Acad Sci USA 97:4802-4807, 2000; Feldman et al., Cancer Res 60:1503-1506, 2000]. In the case of soluble Flt1 ectodomains, Kong et al., [Kong et al., Hum Gene Ther 9:823-833, 1998] have documented the efficacy of adenovirus vector encoded Flt1 when delivered locally, but not systemically, while Takayama et al. have reported systemic antitumor efficacy of adenovirus Flt1, but only against co-injected and not pre-existing tumor burdens [Takayama et al., Cancer Res 60:2169-2177, 2000]. In the case of soluble forms of neuropilin (sNRP), previous studies have shown that a soluble form of neuropilin representing a naturally occurring spliced form of the gene product was able to inhibit the tumorigenic potential of rat prostatic carcinoma cell lines which are themselves engineered to locally express the gene product [Gagnon et al., Proc Natl Acad Sci USA 97:2573-2578, 2000].

Thus, while gene therapy approaches to inhibit VEGF activity and tumor angiogenesis have assumed diverse forms, from intratumoral administration of retroviruses to the local and systemic administration of adenoviruses, these prior studies have not shown effective systemic angiogenesis inhibition using any of the presently available methods. Therefore there exists a need for new strategies to inhibit undesired angiogenesis including tumor-associated angiogenesis.

Traditional cancer treatment methods include cytoreductive therapies that involve administration of ionizing radiation or chemical toxins that kill rapidly dividing cells including cancer cells. Side effects typically result from cytotoxic effects upon normal cells and can limit the use of cytoreductive therapies. A frequent side effect is anemia, a deficiency in the production of red blood cells and result in reduction of oxygen transported by blood cells to the tissues of the body. Side effects, such as anemia, increase morbidity, mortality, and often lead to under-dosing in cancer treatment. A number of studies have shown that correction of anemia, with increased hematocrit, results in marked improvement in various physiologic measures-oxygen utilization [VO2]; muscle strength and function; cognitive and brain electrophysiological function [Wolcott et al. Am J Kidney Dis 14:478-485, 1989]; cardiac function [Wizemann et al. Nephron 64:202-206, 1993; Pascual et al. Clin Nephrol 35:280-287, 1991; Fellner et al. Kidney Int 44:1309-1315, 1993]; sexual function [Shaefer et al. Conitrib Nephrol 76:273-282, 1989]; or quality of life. Evans et al. JAMA 263:825-830, 1990]. Additionally, anemia is often a pre-existing condition in cancer patients resulting from the presence of malignancy, even before commencement of treatment which could further compound anemia. Many clinical investigators have manipulated cytoreductive therapy dosing regimens and schedules to increase dosing for cancer therapy, while limiting damage to bone marrow. One approach involves bone marrow or peripheral blood cell transplants in which bone marrow or circulating hematopoietic progenitor or stem cells are removed before cytoreductive therapy and then reinfused following therapy to restore hematopoietic function. U.S. Pat. No. 5,199,942 describes a method for using GM-CSF, L-3, SF, GM-CSF/IL-3 fusion proteins, erythropoietin (“EPO”) and combinations thereof in autologous transplantation regimens. Clearly, a need thus also exists for cancer therapeutics which not only do not engender anemia, but can also be used simultaneously for its treatment.

The production of red blood cells (RBCs), or erythropoiesis, is stimulated in response to states such as hypoxia, anemia, or high altitude. RBC production, however, cannot proceed unchecked because of potential increased blood viscosity and ischemic end organ damage, as occurs in RBC overproduction states such as polycythemia. Accordingly, erythropoiesis is regulated by a delicate hierarchy of signals including sensing of hypoxia by the transcription factor HIF-1α and the von Hippel-Lindau protein, production of the hormone erythropoietin (EPO) in specialized interstitial cells of the kidney, and stimulation of erythroid precursor formation in the bone marrow [Ebert and Bunn, Blood 94:1864-77, 1999; Ivan et al., Science 292:464-8, 2001; Jaakkola et al., Science 292:468-72, 2001; Zhu and Bunn, Science 292:449-51, 2001].

Erythropoietin is the major known hormonal regulator of RBC production and exerts its effect by binding to the erythropoietin receptor. Activation of the EPO receptor results in several biological effects including stimulation of proliferation, stimulation of differentiation and inhibition of apoptosis [Liboi et al., Proc Natl Sci USA 1990:11351, 1993]. EPO receptor can also be activated by agonists like EPO mutants and analogs, peptides, and antibodies. In addition to EPO, other compounds with erythropoietin-like activity have also been identified. Unfortunately, non-EPO factors capable of stimulating RBC production have not yet been well described in the literature. For example, a molecule identified from a renal cell carcinoma has been reported to have an EPO-like effect on erythropoiesis but is immunologically distinct from EPO [Sytkowski et al., Biochem 18:4095-4099, 1979]. Other stimulators of erythropoiesis include water soluble salts of transition metals [U.S. Pat. No. 5,369,014].

The reduction in RBC mass defined by anemia, often necessitates therapy because of potential physiologic compromise. Blood transfusions represent a commonly used method to treat anemia, such as in the acute blood loss, pre-operative settings, post-radiation or chemotherapy, and chronic anemias, such as from renal failure or destruction of red blood cells by autoantibodies. Severe reductions in erythrocyte levels can be associated with the treatment of various cancers with chemotherapy and radiation and diseases such as AIDS. Anemia is a common side effect of, for example, platinum therapy that is increasingly used to treat solid tumors with the requirement for blood transfusions. Levels of erythrocytes that become too low, for example, hematocrit of less than 25, are likely to produce considerable morbidity and in certain circumstances these levels are life-threatening. In addition, the anemic patients experience significant reduction of the quality of life due to lowered energy levels. The major treatment option is treating the underlying disease. Currently, however, severe acute anemia can only be treated by stimulation of erythropoiesis using EPO or transfusion of red blood cells.

Unfortunately, the over 10 million RBC units annually transfused in the United States engender not-insignificant risks of transfusion reactions, as well as infections including hepatitis viruses and HIV [Goodnough et al., N Engl J Med 340:438-47 1999]. Transfusion is also dependent upon availability of immunologically matching blood products. These infectious and non-infectious risks of transfusions have encouraged the clinical use of erythropoietin (Epo) as a treatment for anemia, with erythropoietin sales exceeding $1 billion annually [Gabrilove, Semin Hematol 37:1-3, 2000].

However, while EPO treatment is considered fairly safe and has relatively few side effects, the treatment often requires several additional weekly injections and adds to patient discomfort. Therefore, there remains a need for additional methods and agents that increase the number of mature red blood cells in anemic individuals by stimulating development and proliferation of cells of the erythroid lineage, including mature red blood cells. There is also a need for methods that can be used in the treatment of anemia associated with the number of cancer treatments, e.g., chemotherapy and radiation.

Furthermore, determining the efficacy or endpoint of a treatment schedule including VEGF or VEGF inhibitors is currently cumbersome. Increased angiogenesis can be determined using immunohistochemical staining of endothelial cells surrounding new blood vessels from a tissue sample. However, this requires taking a biopsy sample which adds to patient discomfort and is not always even possible. In one study, where VEGF-inhibitors were used to treat diabetic maculopathy, the central retinal thickness was determined using a specialized retinal thickness analyzer [Beckendorf et al. 99. Jahrestagung der DOG, Sep. 29-Oct. 2, 2001]. However, this methodology is not generally applicable. One of the limitations in doing antiangiogenic trials is that there are no good surrogate markers for efficacy besides the ultimate clinical response, and there are no well-developed, standardized assays, which is a major limitation of the animal studies of new treatments associated with VEGF, clinical trials as well as the actual treatment methods.

For determining whether the treatment has been effective and when the treatment can be discontinued there exists no simple tests. Therefore, there exists a need for a method to easily and reliably determine an endpoint or efficacy of a treatment including VEGF or VEGF inhibitors.

### SUMMARY OF THE INVENTION

It is therefore an object of the present invention to provide a method for inhibiting undesired angiogenesis including tumor-associated angiogenesis. It is further an object of this invention to provide a method to increase the number of red blood cells or hematocrit in the circulation in subjects in need thereof. Additionally, it is an object of the present invention to provide a method to determine efficacy or endpoint of treatment with VEGF inhibitors.

The truncated, soluble form of Flk1/KDR receptor binds VEGF, therefore, the present invention is useful in treatment of conditions, diseases or disorders associated with VEGF over-expression. In the preferred embodiment the method is used to treat cancer and cancer related anemia. In an alternative embodiment, the method is used to treat cancer in combination with traditional cancer treatments, for example, radiation or chemotherapy. Further, in one embodiment, the truncated, soluble form of Flt-1 is used to treat preexisting tumors and metastatic preexisting tumors.

In one embodiment, the invention provides a method of systemically administering an angiogenesis inhibiting amount of truncated, soluble form of Flk1/KDR to a subject, affected with a condition, disease or disorder associated with VEGF using a nucleic acid encoding the truncated, soluble form of Flk1/KDR. The administration can be performed in the form of a protein in a suitable carrier or in the form of a nucleic acid encoding the protein in a suitable vector

In one embodiment, the truncated, soluble form of Flk1/KDR is administered using a vector. The vectors include viral vectors, liposomes, naked DNA, adjuvant-assisted DNA, gene gun, catheters, chemical conjugates, which have a targeting moiety, and a nucleic acid binding moiety, fusion proteins. The vectors can be chromosomal, non-chromosomal or synthetic. Preferred vectors include viral vectors, fusion proteins and chemical conjugates. Most preferably the viral vector is a gutless adenovirus vector.

In another embodiment the truncated, soluble form of Flk1/KDR is administered as a protein in a pharmaceutically acceptable carrier.

The systemic administration of the truncated, soluble form of Flk1/KDR can be performed intravenously, intramuscularly, intraperitoneally, subcutaneously, through mucosal membranes or via inhalation. Preferably, the truncated, soluble form of Flk1/KDR is administered intravenously.

The subject can be any mammal. Preferably the subject is a murine or a human, most preferably the subject is a human.

In one embodiment, the invention provides a method of increasing hematocrit in a subject in need thereof by administering a hematocrit increasing amount of angiogenesis inhibitor to the subject. Preferably, the angiogenesis inhibitor is a VEGF-blocking or VEGF-inhibiting molecule. Most preferably, the angiogenesis inhibitor is a soluble form of a VEGF receptor including, but not limited to Flt-1, Flt4, neuropilin-1 (NP1), neuropilin-2 (NP2), Flk1/KDR or VEGF-binding fragment thereof in a pharmaceutically acceptable carrier. Preferably the subject in need of increasing the hematocrit levels is or has been treated with radiation or chemotherapy.

In yet another embodiment, the invention provides a method of systemically administering both an angiogenesis inhibiting and hematocrit increasing amount of an angiogenesis inhibitor, preferably a VEGF-inhibitor, most preferably a soluble VEGF receptor or a VEGF-binding fragment thereof, including, but not limited to truncated, Flt-1, Flt-4, neuropilin-1 (NP1), neuropilin-2 (NP2), and Flk1/KDR to a subject, affected with a condition, disease or disorder associated with VEGF and low hematocrit.

In yet another embodiment, the invention provides a method for detecting efficacy of VEGF-inhibitor treatment comprising the steps of providing a first biological sample of a subject before treatment with a VEGF inhibitor, and measuring the hematocrit level in the first sample, and providing a second biological sample from the same individual after treatment with a VEGF inhibitor wherein increased hematocrit level in the second sample indicates effective treatment with a VEGF-inhibitor.

### BRIEF DESCRIPTION OF THE FIGURES

FIG. 1 shows a schematic representation of construction of adenoviruses encoding soluble ectodomains of the VEGF receptors Flk1, Flt1 and neuropilin-1, as well as the anti-angiogenic proteins endostatin and angiostatin, and illustrates insertion of these cDNAs into the E1 region of E3-deleted adenovirus type 5.

FIG. 2 shows a western-blot analysis of adenovirus-expressed anti-angiogenic proteins in mouse plasma. C57B1/6 mice received i.v. injection of 10⁹ particles of the appropriate followed after 2-3 days by Western blot of one microliter of plasma except for Flk1-Fc which was taken at d17 and was a 1:10 dilution. “*” refers to position of transgene products: Flk1-Fc (180 kDa), Flt1(1-3) (53 kDa), ES (20 kDa), AS (55 kDa), sNRP-ABC (120 kDa). Levels in adjacent blots are not comparable because of different ECL exposure times.

FIGS. 3A-D show pharmacokinetics of expression from anti-angiogenic adenoviruses. Plasma from mice infected i.v. with 10⁹ plaque forming units of the appropriate adenovirus was analyzed after the indicated times for expression by ELISA. In FIG. 3A, Flk1-Fe, n=4; FIG. 3B, Flt1, n=4; FIG. 3C, endostatin (ES), n=4; FIG. 3D, angiostatin (AS), n=3).

FIGS. 4A-F demonstrate inhibition of pre-existing tumor growth by anti-angiogenic adenoviruses. In FIG. 4A, C57B1/6 mice were implanted subcutaneously with 10⁶ cells of murine Lewis Lung Carcinoma (LLC). In FIG. 4B mice were implanted subcutaneously with 10⁶ cells of murine T241 Fibrosarcoma. At a tumor volume of 100-150 mm³, tumor-bearing mice received i.v. injection of 10⁹ plaque forming units of the control virus Ad Fc (black bars on the left hand side) or the appropriate anti-angiogenic adenovirus (gray bars on the right hand side) and the tumor volume was calculated after 10-14 days. Tumor size is expressed as percent maximal tumor volume standardized to 100% for Ad-Fc, which did not produce significant inhibition relative to PBS controls. Percent inhibition of animals receiving anti-angiogenic adenoviruses relative to animals injected with the control virus Ad-Fc is calculated. Error bars represent standard error (S.E.) of +/−1. “n” refers to the number of individual mice assayed with each adenovirus. For LLC (FIG. 4A), the number of animals was as follows for Fe and the treatment group: ES n=24,22; AS n=11,9; Flk1-Fc n=18,17; Flt1 n=8,10; sNRP n=8,8. For T241 (FIG. 4B), the number of animals was as follows for Fc and the treatment group: ES n=6,10; AS n=6,7; Flk1-Fc n=24,25; Flt1 n=19,20; sNRP n=7,5. FIG. 4C shows representative growth curves of T241 fibrosarcoma in C57B1/6 mice treated with Ad Flk1-Fc (n=6). FIG. 4D shows representative growth curves of T241 fibrosarcoma in C57B1/6 mice treated with Ad Flt1(1-3) (n=7). In FIG. 4E, representative mice with T241 fibrosarcoma were photographed on day 11 after administration of Ad Fc or Ad Flk1-Fc. FIG. 4F shows suppression of LLC growth by adenoviral delivery of Flk1-Fc or Flt1. Pre-existing tumors of 150 mm³ received i.v. injections of 10⁹ particles of Ad Fc (n=4), Ad Flk1-Fc (n=5) or Ad Flt1(1-3) (n=5), and tumor growth was measured over time. Error bars represent +/−1 standard deviation (S.D.) C57B1/6 mice bearing pre-existing T241 tumors of 100-150 mm³ received 10⁹ plaque forming units i.v. of the appropriate adenoviruses and tumor size was measured over time. Error bars represent +/−1 S.D.

FIGS. 5A-C demonstrate suppression of human tumor xenografts in SCID mice by Ad Flk1-Fc. FIG. 5A shows treatment of BxPC3 human pancreatic carcinoma with Ad Flk1-Fc. CB17 SCID mice bearing pre-existing tumors BxPC3 tumors of 60 mm³ received 10⁹ pfu i.v. of the appropriate adenoviruses and tumor size was measured over time. Error bars represent +/−1 S.D. Fc, n=6; Flk1-Fc, n=7. FIG. 5B shows comparative inhibition of pre-existing BxPC3 tumor growth by anti-angiogenic adenoviruses. Ad Fc and Ad Flk1-Fc mice in FIG. 5B were compared to tumor-bearing mice in the same experiment which received Ad ES (n=7), Ad AS (n=7) or Ad sNRP (n=6). Tumor size is expressed as percent maximal tumor volume standardized to 100% for Ad Fc, which did not produce significant inhibition relative to PBS controls. Error bars represent +/−1 S.E. “n” refers to the number of individual mice assayed with each adenovirus. FIG. 5C shows treatment of human LS174T colon adenocarcinoma in SCID mice with Ad Flk1-Fc. n=5 per group. Error bars indicate +/−1 S.D.

FIG. 5D summarizes the broad-spectrum anti-tumor activity of soluble VEGF receptors Flk1-Fc and Flt1 against a variety of human and murine tumors in subcutaneous, orthotopic and transgenic tumor models.

FIG. 6 demonstrates decreased microvessel density in tumors treated with Ad Flk1-Fc or Ad Flt1(1-3). C57B1/6 mice bearing LLC tumors of approximately 50 mm³ received i.v. injection of 10⁹ plaque forming units of either Ad Fc, Ad Flk1-Fc or Ad Flt1(1-3). Tumors were harvest at a size of 200 mm³ for CD31 immunohistochemistry, magnification and manual quantitation of microvessel density. Error bars represent +/−1 S.D with 4 representative fields counted per condition.

FIG. 7 demonstrates systemic inhibition of corneal angiogenesis by soluble VEGF receptors. The bars illustrate systemic inhibition of corneal neovascularization by Ad Flk1-Fc or Ad Flt1(1-3) in VEGF corneal micropocket assays. C57B1/6 mice received i.v. injection of 10⁹ plaque forming units of the appropriate adenovirus, followed after 2 days by implantation of VEGF-A₁₆₅-containing hydron pellets into the mouse cornea. Five days after pellet implantation, corneal neovascularization was quantitated by slit lamp examination. Results are presented as percent maximal neovascularization relative to the control virus Ad Fc, which was standardized at 100%, and which produced <5% inhibition relative to PBS. Error bars represent +/− standard error (S.E.). The number of eyes examined was as follows for Fc and the treatment group: ES n=13,18; AS n=13,14; Flk1-Fc n=16,15; Flt1n=21,25; sNRP n=10,8. Representative corneas with pre-injection of Ad Fc, Ad Flk1-Fc or Ad Flt1(1-3) were photographed 5 days after pellet implantation. Robust blood vessel ingrowth towards the pellet is noted in Ad Fc but not Ad Flk1-Fc or Ad Flt1(1-3) mice.

FIG. 8 demonstrates that adenoviral delivery of soluble VEGF receptors induces elevations in hematocrit, while soluble extracellular domains of non-VEGF endothelial tyrosine kinase receptors do not. C57B1/6 mice of 10-14 weeks age received i.v. injection of 10⁹ pfu of adenoviruses encoding soluble ectodomains of the following endothelial receptors: Flt1(n=8), Flk1 (n=5), NRP1 (n=2), TIE1 (n=2), TIE2 (n=3), ephrin-B2 (n=2), EphB4 (n=2). Where appropriate, “-Fc” indicates a C-terminal IgG2α Fc fusion. PBS or Ad Fc was injected as controls. Western blotting or ELISA were performed on serum of mice at day 2 post-infection to confirm expression of the respective transgenes. At day 14 post-injection, hematocrit was determined by microcapillary centrifugation of whole blood obtained by retroorbital puncture. Error bars indicate one standard deviation (S.D.).

FIGS. 9A-B demonstrate dose- and time-dependent increases in hematocrit following soluble VEGFR treatment. Sixteen week-old C57B1/6 mice (n=4) received i.v. injection of 10⁹−3×10⁷ pfu of Ad Fc, Ad Flt1 (FIG. 9A) or Ad Flk1-Fc (FIG. 9B), and serial determinations of hematocrit by microcapillary spin method were performed at the indicated times. Plasma from day 3 phlebotomy was also analyzed by ELISA to quantitate systemic expression of Flt1 and Flk1-Fc and these values are listed next to the appropriate curves.

FIG. 10 demonstrates selective increases in RBC, but not WBC or platelets, following soluble VEGFR treatment. Fourteen week-old male C57B1/6 mice received i.v. injection of 10⁹ pfu of Ad Flt1, Flk1-Fc or Fc, followed after 14 days by automated CBC determination of WBC, RBC and platelet number.

FIG. 11 indicates that arterial oxygen concentration is not altered in mice treated with soluble Flt1. Fourteen week old C57B1/6 mice received i.v. injection of 10⁹ pfu of either Ad Fc or Ad Flt1. After 13 days, indwelling arterial catheters were inserted into the carotid artery followed the next day by resting sampling of arterial blood for hematocrit (microcapillary spin method) and arterial blood gas analysis (automated). The partial pressures of arterial oxygen (pO₂) are boxed.

FIG. 12 indicates that BUN/creatinine ratios, a measure of hydration status, are unaltered in soluble VEGFR-treated mice. Ten week-old C57B1/6 mice received i.v. injection of 10⁹ pfu of Ad Fc (n=5), Ad Flt1 (n=4), Ad Flk1-Fc (n=5) or PBS (n=4) as appropriate, followed after 14 days by sampling of plasma for automated detection of blood urea nitrogen (BUN) or creatinine (Cr), and whole blood for hematocrit.

FIG. 13 shows increased reticulocytosis in mice after adenoviral delivery of Flt1 or Flk1-Fc, but not when only Fc was used. Fourteen week-old male C57B1/6 mice received i.v. injection of 10⁹ pfu of Ad Flt1, Flk1-Fc or Fc, followed after 14 days by reticulin staining of peripheral blood, and manual counting of reticulocytes. Reticulocyte count is shown as defined by % (reticulocytes/non-reticulocytes) and was determined after the indicated times following adenovirus administration.

FIGS. 14A-B show a summary of induction of Ter119(+) CD45(−) erythroid precursors by soluble VEGF receptors in splenocytes (FIG. 14A) and in bone marrow cells (FIG. 14B). Ter119(+) CD45(−) cells as a percentage of total cells are indicated on the Y-axis. This experiment demonstrates induction of Ter119(+) CD45(−) erythroid precursors following soluble VEGFR-mediated VEGF blockade. C57B1/6 mice of 14-16 weeks of age received i.v. injection of 10⁹ pfu of Ad Flt1, Flk1-Fc or Fc, followed after 14 days by FACS analysis of bone marrow cells or splenocytes using anti-Ter119-PE and anti-CD45-FITC antibody conjugates.

### DETAILED DESCRIPTION OF THE INVENTION

The present invention is based upon the surprising finding that a viral construct encoding a truncated, soluble form of Flk1/KDR receptor which is administered systemically can reach therapeutically effective antiangiogenic levels to treat large pre-existing tumors. Comparison with constructs encoding other antiangiogenic proteins such as endostatin, angiostatin, and neuropilin shows that the truncated, soluble form of Flk1/KDR is superior to them. Some prior studies have suggested that soluble Flt1, which binds VEGF with much higher affinity than Flk1/KDR, reduces angiogenesis and would be preferred over the weaker and ineffective angiogenesis inhibitor Flk1/KDR. However, the present invention demonstrates that Flt1 is associated with significant toxicity at high doses. In contrast, the truncated, soluble form of Flk1/KDR inhibited angiogenesis with similar efficacy than Flt1 but without the toxic side effects, making the use of the truncated, soluble form of Flk1/KDR unexpectedly a preferred method of angiogenesis inhibition. The present invention also demonstrates that lower amounts of viral construct encoding soluble Flt1 can still elicit biological responses.

The method of the present invention is also based upon the surprising finding that administration of angiogenesis inhibitors in an individual results in increased number of red blood cells or hematocrit level, thereby providing an unexpected treatment of anemia. The method of the present invention includes use of not only one angiogenesis inhibitor but also a combination of various angiogenesis inhibitors to increase hematocrit levels in individuals affected with anemia. The angiogenesis inhibitor may be any angiogenesis inhibitor, including but not limited to inhibitors such as tyrosine kinase inhibitors, TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, inhibitor of a basic fibroblast derived growth factor (bFGF), such as a soluble bFGF receptor, transforming growth factor beta, interferon alfa, an inhibitor of epidermal-derived growth factor, an inhibitor of platelet derived growth factor, an intergrin blocker, interleukin-12, troponin-1, and an antibody to VEGF. In a preferred embodiment, the angiogenesis inhibitor is a VEGF inhibitor, such as a small molecule that is capable of blocking VEGF function, an antibody to an immunogenic epitope of VEGF or a soluble VEGF-receptor including but not limited to Flt1, Flt4, neuropilin-1 (NP1), neuropilin-2(NP2), Flk1/KDR, or a combination of different VEGF inhibitors (for additional antiangiogenic compounds, see below).

The truncated, soluble form of Flk1/KDR receptor binds VEGF, therefore the present invention is useful in treatment of conditions, diseases or disorders associated with VEGF over-expression. The invention is also useful in treating conditions associated with both VEGF over-expression and anemia. In the preferred embodiment the method is used to treat cancer. In an alternative embodiment, the method is used in combination with other angiogenesis inhibitors. In one embodiment the truncated, soluble form of Flk1/KDR construct of the present invention is used in combination with more traditional cancer treatments such as radiation or chemotherapy to supplement the treatment of cancer and simultaneously alleviate anemia associated with radiation or chemotherapy.

The invention is further based upon a discovery that hematocrit levels can be used as indicators of efficient VEGF-inhibitor related therapy.

As used herein, the term “truncated, soluble form” of Flk1/KDR or Flt1 receptor means a receptor molecule encoding extracellular portions of Flk1/KDR or Flt1 excluding membrane bound and intracellular regions, said truncated soluble receptor molecule being capable of binding to and inhibiting the activity of VEGF fused to a C terminal IgG2α antibody Fc fragment (either human or murine) which increases stability. Preferably, the truncated, soluble form of Flk1/KDR or Flt1 receptor of the present invention consists of amino acid sequences derived from Ig-like domains from the extracellular ligand-binding region of the Flk1/KDR or Flt1 receptor.

As used herein, the term “VEGF receptor” means a receptor, a modified or mutated receptor, or a fragment of the receptor or a modified or mutated receptor, that is capable of binding VEGF. Such receptors include, but are not limited to, Flt1, Flt4, NP1, NP2, and Flk1/KDR. The VEGF receptor or a soluble form of a VEGF receptor can be used alone or it can be fused to a C terminal IgG2α antibody Fc fragment (either human or murine) which increases stability.

The term “Flk1/KDR receptor” as used herein is meant to encompass both human and murine homologues of the receptor as well as functional Flk1/KDR receptors that have been genetically engineered to contain one or more point mutations which may or may not alter the affinity of the receptor to its ligand.

The term “Flt1receptor” as used herein is also meant to encompass both human and murine homologues of the receptor as well as functional Flt1 receptors that have been genetically engineered to contain one or more point mutations which may or may not alter the affinity of the receptor to its ligand.

The term “VEGF-binding”, “VEGF-blocking”, and “VEGF-inhibiting” molecule are used interchangeably in the present application and are meant to include molecules or compounds or agents that are capable of preventing or inhibiting VEGF mediated signaling pathways. These compounds include nucleic acids, modified nucleic acids, small organic and inorganic molecules, proteins and modified proteins, and antibodies. Several assays are known to one skilled in the art to determine whether an agent inhibits VEGF signaling. Examples of such assays include, but are not limited to growth inhibition assay, cord formation assay and cell migration assay. Examples of reference compounds that can be used in the assays are TNP-470 (NSC 642492) and paclitaxel (NSC 125973) (http://dtp.nci.gov/).

A short exemplary description of the VEGF inhibitor determining assays that is not to be construed as a limiting description of such assays follows:

In growth inhibition assay HUVEC (1.5×10³) are plated in a 96-well plate in 100 μl of EBM-2 (Clonetic #CC3162). After 24 h (day 0), the test compound (100 μl) is added to each well at 2× the desired concentration (5-7 concentration levels) in EBM-2 medium. On day 0, one plate is stained with 0.5% crystal violet in 20% methanol for 10 minutes, rinsed with water, and air-dried. The remaining plates are incubated for 72 h at 37° C. After 72 h, plates are stained with 0.5% crystal violet in 20% methanol, rinsed with water and air-dried. The stain is eluted with 1:1 solution of ethanol:0.1M sodium citrate (including day 0 plate), and absorbance is measured at 540 nm with an ELISA reader (Dynatech Laboratories). Day 0 absorbance is subtracted from the 72 h plates and data is plotted as percentage of control proliferation (vehicle treated cells). IC₅₀ (compound concentration causing 50% inhibition) is calculated from the plotted data.

In cord formation assay Matrigel™ (60 μl of 10 mg/ml; Collaborative Lab #35423) is placed in each well of an ice-cold 96-well plate. The plate is allowed to sit at room temperature for 15 minutes then incubated at 37° C. for 30 minutes to permit the matrigel to polymerize. In the mean time, HUVEC are prepared in EGM-2 (Clonetic #CC3162) at a concentration of 2×10⁵ cells/ml. The test compound is prepared at 2× the desired concentration (5 concentration levels) in the same medium. Cells (500 μl) and 2× test compound (500 μl ) is mixed and 200 μl of this suspension are placed in duplicate on the polymerized Matrigel™. After 24 h incubation, triplicate pictures are taken for each concentration using a Bioquant Image Analysis system. Effect of the compound (IC₅₀) is assessed compared to untreated controls by measuring the length of cords formed and number of junctions.

In cell migration assay migration is assessed using the 48-well Boyden chamber and 8 μm pore size collagen-coated (10 μg/ml rat tail collagen; Collaborative Laboratories) polycarbonate filters (Osmonics, Inc.). The bottom chamber wells receive 27-29 μl of DMEM medium alone (baseline) or medium containing chemo-attractant (e.g. bFGF, VEGF or Swiss 3T3 cell conditioned medium). The top chambers receive 45 μl of HUVEC cell suspension (1×10⁶ cells/ml) prepared in DMEM+1% BSA with or without test compound. After 5 h incubation at 37° C., the membrane is rinsed in PBS, fixed and stained in Diff-Quick solutions. The filter is placed on a glass slide with the migrated cells facing down and cells on top are removed using a Kimwipe. The testing is performed in 4-6 replicates and five fields are counted from each well. Negative unstimulated control values are subtracted from stimulated control and compound treated values and data is plotted as mean migrated cell ± S.D. IC₅₀ is calculated from the plotted data.

“Immunoglobulin-like domain” or “Ig-like domain” refers to each of the seven independent and distinct domains that are found in the extracellular ligand-binding region of the Flt1and Flk1/KDR receptors. Ig-like domains are generally referred to by number (see, e.g., U.S. Pat. No. 5,952,199). As used herein, the term “Ig-like domain” is intended to encompass not only the complete wild-type domain, but also insertional, deletional and substitutional variants thereof which substantially retain the functional characteristics of the intact domain. It will be readily apparent to those of ordinary skill in the art that numerous variants of the Ig-like domains of the Flk1/KDR receptor can be obtained which will retain substantially the same functional characteristics as the wild type domain.

“Soluble” as used herein with reference to the receptor proteins used in the present invention is intended to mean a receptor protein which is not fixed to the surface of cells via a transmembrane domain. As such, soluble forms of a receptor protein of the present invention, while capable of binding to and inactivating VEGF, do not comprise a transmembrane domain and thus generally do not become associated with the cell membrane of cells in which the molecule is expressed. A soluble form of the receptor exerts an inhibitory effect on the biological activity of the VEGF protein by binding to VEGF, thereby preventing it from binding to its natural receptors present on the surface of target cells.

The angiogenesis inhibitor, including, the soluble VEGF receptors, such as truncated, soluble form of Flk1/KDR or Flt1 can be administered as a recombinant protein, recombinant fusion protein or transferring a gene encoding such angiogenesis inhibitor in a vector and delivering such vector encoding the angiogenesis inhibitor into a subject in need thereof. The term “vector” includes viral vectors, liposomes, naked DNA, adjuvant-assisted DNA, gene gun, catheters, etc. The term “vector” also encompasses chemical conjugates such as described in WO 93/04701, which have a targeting moiety (e.g. a ligand to a cellular surface receptor), and a nucleic acid binding moiety (e.g. polylysine), viral vector (e.g. a DNA or RNA viral vector), fusion proteins such as described in PCT/US 95/02140 (WO 95/22618) which is a fusion protein containing a target moiety (e.g. an antibody specific for a target cell) and a nucleic acid binding moiety (e.g. a protamine), plasmids, phage, etc. The vectors can be chromosomal, non-chromosomal or synthetic.

The gene delivery or transfer methods using a vector fall into three broad categories: (1) physical (e.g., electroporation, direct gene transfer and particle bombardment), (2) chemical (e.g. lipid-based carriers and other non-viral vectors) and (3) biological (e.g. virus derived vectors). For example, non-viral vectors such as liposomes coated with DNA may be directly injected intravenously into the patient. It is believed that the liposome/DNA complexes are concentrated in the liver where they deliver the DNA to macrophages and Kupffer cells.

Gene transfer methodologies can also be described by delivery site. Fundamental ways to deliver genes include ex vivo gene transfer, in vivo gene transfer, and in vitro gene transfer. In ex vivo gene transfer, cells are taken from the patient and grown in cell culture. The DNA is transfected into the cells, the transfected cells are expanded in number and then reimplanted in the patient. In vitro gene transfer, the transformed cells are cells growing in culture, such as tissue culture cells, and not particular cells from a particular patient. These “laboratory cells” are transfected, the transfected cells are selected and expanded for either implantation into a patient or for other uses. In vivo gene transfer involves introducing the DNA into the cells of the patient when the cells are within the patient. All three of the broad based categories described above may be used to achieve gene transfer in vivo, ex vivo, and in vitro.

Mechanical (i.e. physical) methods of DNA delivery can be achieved by direct injection of DNA, such as microinjection of DNA into germ or somatic cells, pneumatically delivered DNA-coated particles, such as the gold particles used in a “gene gun,” and inorganic chemical approaches such as calcium phosphate transfection. It has been found that physical injection of plasmid DNA into muscle cells yields a high percentage of cells which are transfected and have a sustained expression of marker genes. The plasmid DNA may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Particle-mediated gene transfer may also be employed for injecting DNA into cells, tissues and organs. With a particle bombardment device, or “gene gun,” a motive force is generated to accelerate DNA-coated high density particles (such as gold or tungsten) to a high velocity that allows penetration of the target organs, tissues or cells. Electroporation for gene transfer uses an electrical current to make cells or tissues susceptible to electroporation-mediated gene transfer. A brief electric impulse with a given field strength is used to increase the permeability of a membrane in such a way that DNA molecules can penetrate into the cells. The techniques of particle-mediated gene transfer and electroporation are well known to those of ordinary skill in the art.

Chemical methods of gene therapy involve carrier mediated gene transfer through the use of fusogenic lipid vesicles such as liposomes or other vesicles for membrane fusion. A carrier harboring a DNA of interest can be conveniently introduced into body fluids or the bloodstream and then site specifically directed to the target organ or tissue in the body. Liposomes, for example, can be developed which are cell specific or organ specific. The foreign DNA carried by the liposome thus will be taken up by those specific cells. Injection of immunoliposomes that are targeted to a specific receptor on certain cells can be used as a convenient method of inserting the DNA into the cells bearing the receptor. Another carrier system that has been used is the asialoglycoprotein/polylysine conjugate system for carrying DNA to hepatocytes for in vivo gene transfer.

Transfected DNA may also be complexed with other kinds of carriers so that the DNA is carried to the recipient cell and then resides in the cytoplasm or in the nucleoplasm of the recipient cell. DNA can be coupled to carrier nuclear proteins in specifically engineered vesicle complexes and carried directly into the nucleus.

Carrier mediated gene transfer may also involve the use of lipid-based proteins which are not liposomes. For example, lipofectins and cytofectins are lipid-based positive ions that bind to negatively charged DNA, forming a complex that can ferry the DNA across a cell membrane. Another method of carrier mediated gene transfer involves receptor-based endocytosis. In this method, a ligand (specific to a cell surface receptor) is made to form a complex with a gene of interest and then injected into the bloodstream; target cells that have the cell surface receptor will specifically bind the ligand and transport the ligand-DNA complex into the cell.

Biological gene therapy methodologies usually employ viral vectors to insert genes into cells. The term “vector” as used herein in the context of biological gene therapy means a carrier that can contain or associate with specific polynucleotide sequences and which functions to transport the specific polynucleotide sequences into a cell. The transfected cells may be cells derived from the patient's normal tissue, the patient's diseased tissue, or may be non-patient cells. Examples of vectors include plasmids and infective microorganisms such as viruses, or non-viral vectors such as the ligand-DNA conjugates, liposomes, and lipid-DNA complexes discussed above.

Viral vector systems which may be utilized in the present invention include, but are not limited to (a) adenovirus vectors; (b) retrovirus vectors; (c) adeno-associated virus vectors; (d) herpes simplex virus vectors; (e) SV 40 vectors; (f) polyoma virus vectors; (g) papilloma virus vectors; (h) picarnovirus vectors; (i) vaccinia virus vectors; and (j) a helper-dependent or gutless adenovirus. In the preferred embodiment the vector is an adenovirus or adeno-associated virus. In the most preferred embodiment the vector is a gutless adenovirus.

For example, a helper-dependent, or gutless, adenoviral vector (hdAd) can promote stable transgene expression in peripheral organs, including the liver. The gutless vectors are completely devoid of viral proteins. They are constructed by using so called helper viruses that provide the necessary proteins in trans for the packing of a vector devoid of viral genes [Parks et al., Proc Natl Acad Sci USA 93:13565-13570, 1996; Hardy et al., J Virol 71:1842-1849, 1997]. Using helper-dependent vectors a long term expression may be achieved with only one injection if such is desired. Additionally, if several injections are considered to be necessary to achieve sufficient plasma concentrations of Flk1/KDR, booster injections may be used. To avoid possible immune responses to the viral capsid proteins, vectors of different serotypes are preferred [Kass-Eisler et al., Gene Ther 3:154-162, 1996; Mastrageli et al., Hum Gene Ther 7:79-87, 1996]. A gutless vector may be produced, for example, as described in [Morral et al., Proc Natl Acad Sci USA 96:12816-12821, 1999].

The viral construct according to the present invention encoding an angiogenesis inhibitor, for example, truncated, soluble Flk1/KDR receptor can be used for the inhibition of VEGF mediated activity including angiogenesis and tumor cell motility or alternatively for increasing the hematocrit level. For intravenous applications, the inhibitor is used at an amount of 1×10⁵−2×10¹¹ plaque forming units (pfu).

Administration of the angiogenesis inhibitor, for example a soluble VEGF receptor such as Flk1/KDR, can be combined with a therapeutically effective amount of another molecule which negatively regulates angiogenesis which may be, but is not limited to VEGF inhibitors such as antibodies against VEGF or antigenic epitopes thereof, and soluble VEGF receptors such as Flt-1, Flk-1/KDR, Flt-4, neuropilin-1 and -2 (NP1 and NP2); TNP-470; PTK787/ZK 222584 (1-[4chloroanilino]-4-[4-pyridylmethyl]phthalazine succinate)[Novartis International AG, Basel, Switzerland]; VEGF receptor inhibitors, such as SU5416, or antibodies against such receptors such as DC101 [ImClone Systems, Inc., NY]; tyrosine kinase inhibitors; prolactin (16-Kd fragment), angiostatin (38-kD fragment of plasminogen), endostatin, basic fibroblast derived growth factor (bFGF) inhibitors such as a soluble bFGF receptor; transforming growth factor beta; interferon alfa; epidermal-derived growth factor inhibitors; platelet derived growth factor inhibitors; an intergrin blocker, interleukin-12; troponin-1; 12-lipoxygenase (LOX) inhibitors, such as BHPP (N-benzyl-N-hydroxy-5-phenylpentanamide)[Nie et al. Blood 95:2304-2311]; platelet factor 4; thrombospondin-1; tissue inhibitors of metalloproteases such as TIMP1 and TIMP2; transforming growth factor beta; interferon alfa; protamine; combination of heparin and steroids; and steroids such as tetrahydrocortisol; which lack gluco- and mineral-corticoid activity; angiostatin; phosphonic acid agents; anti-invasive factor; retinoic acids and derivatives thereof; paclitaxel [U.S. Pat. No. 5,994,341]; interferon-inducible protein 10 and fragments and analogs of interferon-inducible protein 10; medroxyprogesterone; sulfated protamine; prednisolone acetate; herbimycin A; peptide from retinal pigment epithelial cell; sulfated polysaccharide; and phenol derivatives; isolated body wall of a sea cucumber, the isolated epithelial layer of the body-wall of the sea cucumber, the flower of the sea cucumber, their active derivatives or mixtures thereof; thalidomide and various related compounds such as thalidomide precursors, analogs, metabolites and hydrolysis products; 4 kDa glycoprotein from bovine vitreous humor; a cartilage derived factor, human interferon-alpha; ascorbic acid ethers and related compounds; sulfated polysaccharide DS 4152; and a synthetic fumagillin derivative, AGM 1470. In the preferred embodiment of the present invention, the angiogenesis inhibitor is a VEGF inhibitor. Most preferably the angiogenesis inhibitor is truncated, soluble form of a VEGF receptor.

The truncated, soluble form of Flk/KDR of the invention may also be combined with chemotherapeutic agents or radiation therapy and can be administered before, during or after chemotherapy or radiotherapy treatment.

A preferred embodiment of the present invention relates to a method of inhibiting angiogenesis associated with solid tumors to inhibit or prevent further tumor growth and eventual metastasis and to reduce the size of a preexisting tumor.

Any solid tumor containing cells that express VEGF or its receptors will be a potential target for treatment. Examples, but by no means listed as a limitation, of solid tumors which will be particularly vulnerable to gene therapy applications are (a) neoplasms of the central nervous system such as, but again not necessarily limited to glioblastomas, astrocytomas, neuroblastomas, meningiomas, ependymomas; (b) cancers of hormone-dependent, tissues such as prostate, testicles, uterus, cervix, ovary, mammary carcinomas including but not limited to carcinoma in situ, medullary carcinoma, tubular carcinoma, invasive (infiltrating) carcinomas and mucinous carcinomas; (c) melanomas, including but not limited to cutaneous and ocular melanomas; (d) cancers of the lung which at least include squamous cell carcinoma, spindle carcinoma, small cell carcinoma, adenocarcinoma and large cell carcinoma; and (e) cancers of the gastrointestinal system such as esophageal, stomach, small intestine, colon, colorectal, rectal and anal region which at least include adenocarcinomas of the large bowel.

For purposes herein, the “therapeutically effective amount” of angiogenesis inhibitor, for example, truncated, soluble form of Flk1/KDR receptor protein is an amount that is effective to either prevent, lessen the worsening of, alleviate, or cure the treated condition, in particular that amount which is sufficient to reduce or inhibit the proliferation of vascular endothelium or increase hematocrit or both in vivo. The therapeutically effective amount of the angiogenesis inhibitor, for example, truncated, soluble form of Flk1/KDR receptor protein, to be administered will be governed by considerations such as the disorder being treated, the particular mammal being treated, the clinical condition of the individual subject, the cause of the disorder, the site of delivery of the angiogenesis inhibitor, for example, truncated, soluble form of Flk1/KDR receptor protein, the method of administration, the scheduling of administration, and other factors known to medical practitioners.

An effective amount to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. Typically, the clinician will administer until a dosage is reached that achieves the desired effect.

Diseases, disorders, or conditions, associated with abnormal angiogenesis or neovascularization where VEGF expression is abnormal, and can be treated with the method of the present invention include, but are not limited to retinal neovascularization, tumor growth, hemangioma, solid tumors, leukemia, metastasis, psoriasis, neovascular glaucoma, diabetic retinopathy, arthritis, endometriosis, and retinopathy of prematurity (ROP). The method of the present invention can also be used to treat anemia which can be caused by several reasons including, but not limited to radiation and chemotherapy, autoimmune disorders, kidney disorders, and bleeding disorders.

VEGF regulation of adult erythropoiesis has not previously been suspected. Phenotypes of knockout animals suggest that embryonic hematopoiesis actually requires Flk1 (and by inference VEGF) function [Shalaby et al., Nature 376:62-6, 1995] which is in contrast to the our data in adult mice, where inhibition of Flk1/VEGF function actually increases erythropoiesis without alteration in other hematopoietic lineages. In neonatal mice (1-7 days post-partum), administration of a soluble Flt1 receptor produced developmental hypoplasia of heart and lung and lethality [Gerber et al., Development 126:1149-59, 1999]. These neonates incidentally exhibited very mild polycythemia which was presumed secondary to hypoxemia from heart/lung hypoplasia, which clearly does not occur in our studies using fully developed adult mice.

A number of angiogenesis inhibitors have been identified. The angiogenesis inhibitors useful in the present method of increasing the hematocrit include, but are not limited to, VEGF inhibitors such as antibodies against VEGF or antigenic epitopes thereof, and soluble VEGF receptors such as Flt-1, Flk-1/KDR, Flt-4, neuropilin-1 and -2 (NP1 and NP2); TNP-470; PTK787/ZK 222584 (1-[4chloroanilino]-4-[4-pyridylmethyl]phthalazine succinate)[Novartis International AG, Basel, Switzerland]; VEGF receptor inhibitors, such as SU5416, or antibodies against such receptors such as DC101 [ImClone Systems, Inc., NY]; tyrosine kinase inhibitors; prolactin (16-Kd fragment), angiostatin (38-kD fragment of plasminogen), endostatin, basic fibroblast derived growth factor (bFGF) inhibitors such as a soluble bFGF receptor; transforming growth factor beta; interferon alfa; epidermal-derived growth factor inhibitors; platelet derived growth factor inhibitors; an intergrin blocker; interleukin-12; troponin-1; 12-lipoxygenase (LOX) inhibitors, such as BHPP (N-benzyl-N-hydroxy-5-phenylpentanamide)[Nie et al. Blood 95:2304-2311]; platelet factor 4; thrombospondin-1; tissue inhibitors of metalloproteases such as TIMP1 and TIMP2; transforming growth factor beta; interferon alfa; protamine; combination of heparin and steroids; and steroids such as tetrahydrocortisol; which lack gluco- and mineral-corticoid activity; angiostatin; phosphonic acid agents; anti-invasive factor; retinoic acids and derivatives thereof; paclitaxel [U.S. Pat. No. 5,994,341]; interferon-inducible protein 10 and fragments and analogs of interferon-inducible protein 10; medroxyprogesterone; sulfated protamine; prednisolone acetate; herbimycin A; peptide from retinal pigment epithelial cell; sulfated polysaccharide; and phenol derivatives; isolated body wall of a sea cucumber, the isolated epithelial layer of the body-wall of the sea cucumber, the flower of the sea cucumber, their active derivatives or mixtures thereof; thalidomide and various related compounds such as thalidomide precursors, analogs, metabolites and hydrolysis products; 4 kDa glycoprotein from bovine vitreous humor; a cartilage derived factor; human interferon-alpha; ascorbic acid ethers and related compounds; sulfated polysaccharide DS 4152; and a synthetic fumagillin derivative, AGM 1470. In the preferred embodiment of the present invention, the angiogenesis inhibitor is a VEGF inhibitor. Most preferably the angiogenesis inhibitor is truncated, soluble form of a VEGF receptor.

One embodiment of the invention, a method of increasing hematocrit, is especially useful in treating conditions associated with both anemia and a condition, disease or disorder associated with increased angiogenesis. In the preferred embodiment the method is used to treat cancer and cancer treatment related anemia. In one embodiment, the method is used in increasing hematocrit levels in combination with traditional cancer treatments, for example, radiation or chemotherapy. In another embodiment, the method is used to treat anemia alone in individuals suffering from anemia without cancer.

The angiogenesis inhibitor, such as truncated, soluble form of Flk1/KDR receptor protein can be incorporated into a pharmaceutical composition suitable for administration. Such compositions typically comprise the nucleic acid molecule, protein, antibody, or other active small molecule and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical composition is formulated to be compatible with its intended route of administration. Examples of routes of administration include parenteral, e.g., intravenous, intradermal, subcutaneous, or oral. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion For intravenous administration, suitable carriers include physiological saline, bacteriostatic water, Cremophor EL.TM. (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the angiogenesis inhibitor in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients, e.g., from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation (Mountain View, Calif.) or Nova Pharmaceuticals, Inc (Lake Elsinore, Calif.). Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active angiogenesis inhibiting compound and the particular therapeutic effect to be achieved, and the limitations inherent in the art of compounding such an active compound for the treatment of individuals.

Our finding that truncated, soluble forms of Flk1 and Flt1 possessed significantly more potent anti-tumor activity than angiostatin or endostatin when delivered via gene transfer was unexpected and is of particular interest in light of previous reports of the extremely potent anti-tumor effects of endostatin and angiostatin delivered via conventional protein administration [O'Reilly et al., Cell 79:315-328, 1994; O'Reilly et al., Nat Med 2:689-692, 1996; O'Reilly et al., Cell 88:277-85, 1997]. The reasons for this important discrepancy are not currently clear. Although the serum levels of angiostatin and endostatin achieved in the previous studies which reported frank tumor regression were not measured [Id.], it is highly likely that the levels of the proteins obtained after adenoviral mediated gene transfer are far greater. In addition, while differences in protein structure, folding, or post-translational processing between the conventionally produced molecules and those produced via gene transfer could account for differences in their bioactivity, mass spectroscopy and N-terminal sequencing of at least the vector-produced endostatin isolated from mouse serum suggests its integrity. Moreover, as indicated earlier, the adenovirus-produced endostatin exhibits motility-inhibiting properties comparable to that of recombinant endostatin produced in yeast, baculovirus or myeloma cells in matrigel assays. Taken together, the data suggests that, at a minimum, endostatin or angiostatin will not be as easily utilizable as soluble VEGF receptors in conventional single-injection adenoviral strategies aimed at the systemic delivery of protein, and may require more innovative approaches with different vector systems, modified transgenes or alternative routes of administration.

Although several previous reports had also documented the anti-tumor effects of vector-mediated delivery of angiostatin, endostatin, soluble Flt1 ectodomains, and soluble neuropilin (sNRP) domains, [Takayama et al., Cancer Res 60:2169-2177, 2000; Griscelli et al., Proc Natl Acad Sci USA 95:6367-6372, 1998; Blezinger et al., Nat Biotechnol 17:343-348, 1999; Chen et al., Cancer Res 59:3308-3312, 1999; Sauter et al., Proc Natl Acad Sci USA 97:4802-4807, 2000; Feldman et al., Cancer Res 60:1503-1506, 2000], the ability of the gene products delivered by gene therapy to provide for the potent inhibition of large (>100 mm³) aggressive pre-existing tumors, such as LLC, had not previously been demonstrated.

For example, systemic gene therapy with angiostatin has not been well documented to strongly suppress pre-existing tumor growth although it has been shown that tumor lines stably transfected with angiostatin cDNA exhibit impaired tumor growth, [Griscelli et al., Proc Natl Acad Sci USA 95:6367-6372, 1998; Blezinger et al., Nat Biotechnol 17:343-348, 1999; Chen et al., Cancer Res 59:3308-3312, 1999]. Additionally, no strong activity against pre-existing tumors has been reported, although several studies report the inhibition of tumor growth and metastases in mice after vector-mediated delivery of endostatin, [Blezinger et al., Nat Biotechnol 17:343-348, 1999; Chen et al., Cancer Res 59:3308-3312, 1999; Sauter et al., Proc Natl Acad Sci USA 97:4802-4807, 2000].

In the case of soluble Flt-1 ectodomains, Kong et al. [Hum Gene Ther 9:823-833, 1998] have documented the efficacy of adenovirus vector encoded Flt when delivered locally, but not systemically, while Takayama et al. [Cancer Res 60:2169-2177, 2000] have reported systemic antitumor efficacy of adenovirus Flt, but only against co-injected and not pre-existing tumor burdens. In this latter case, the inability to observe significant activity against pre-existing tumors may have resulted from insufficient production of Flt ectodomains, as our preliminary dosing studies suggest that high levels of gene product (>2 μl/ml) may be necessary for activity against preexisting tumors of >100 mm³. In the case of soluble forms of neuropilin (sNRP), previous studies have shown that a soluble form of neuropilin representing a naturally occurring spliced form of the gene product was able to inhibit the ability of rat prostatic carcinoma cell lines engineered to express the gene product to grow as tumors [Gagnon et al., Proc Natl Acad Sci USA 97:2573-2578, 2000]. The inability of our Ad sNRP to inhibit tumor growth could reflect either the stringency of the tumor models used our study or the use of a sub-optimal soluble form of NRP (the sNRP gene used in the current studies differs from that used in previous studies in that the “C” domain is included). It is noteworthy that sNRP binds to regions of VEGF encoded by exon 7 [Soker et al., Cell 92:735-745, 1998; Soker et al., J Biol Chem 271:5761-5767, 1996] while Flk1 and Flt1 bind to more N-terminal domains of VEGF [Keyt et al., J Biol Chem 271:5638-5646, 1996].

In addition to identifying candidate gene products of potential use in cancer therapy, the work presented here also represent the first comparative study of systemically administered anti-angiogenic agents against ocular angiogenesis. Small molecule inhibitors of the Flk1/KDR kinase domain, direct intraocular injection of soluble VEGF receptors, or adenoviral production of soluble Flt-1 have been previously shown to inhibit experimental retinal vascularization [Aiello et al., Proc Natl Acad Sci USA 92:10457-10461, 1995; Honda et al., Gene Ther 7:978-985, 2000; Ozaki et al., Am J Pathol 156:697-707, 2000]. Potentially, a variety of conditions accompanied by pathologic eye angiogenesis, such as diabetic retinopathy, macular degeneration, retinal ischemia and ocular melanomas [Aiello, Ophthalmic Res 29:354-362, 1997; Aiello, Curr Opin Ophthalmol 8:19-31, 1997] could benefit from the sustained delivery afforded by single injection dosing of gene transfer vectors.

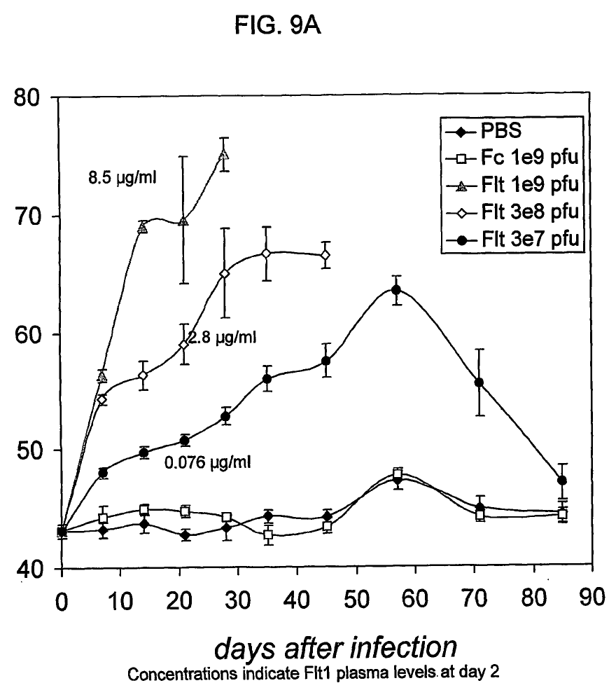

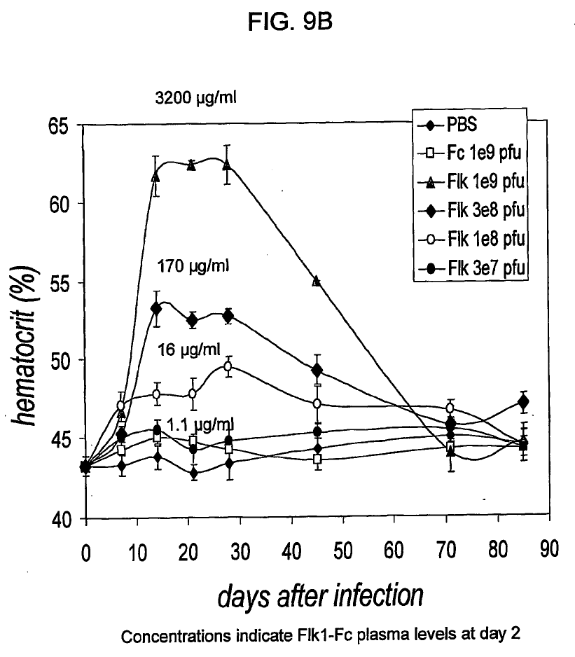

The expression levels we have achieved likely represent a theoretical “maximum” which reflects the inherent pharmacokinetic properties governing the circulating levels of each proteins that can be achieved via gene transfer. As such, the results provide important practical information regarding which anti-angiogenic gene products are most likely to be therapeutically effective when delivered via gene therapy. In addition to the need to evaluate the use of vector systems which can provide for the sustained high level expression of genes in vivo, such as the ‘gutless’ adenoviral vectors [Mountain, Trends Biotechnol 18:119-128, 2000], considerably more effort will need to be paid to the issue of the safety and long-term sequelae of systemic, soluble receptor-mediated VEGF inhibition in adult organisms. In this regard, we have observed that while non-tumor-bearing animals injected with Ad Flk1-Fc and viruses encoding endostatin, angiostatin and sNRP remained grossly asymptomatic for >1 year, approximately 30% of animals injected with maximal doses (10⁹ pfu) of Ad Flt1(1-3) develop ascites after 22-28 days followed by frequent mortality despite a several log lower serum concentration of Flt than Flk1-Fc (unpublished results). This toxicity is titratable as animals receiving lower doses (3×10⁷ pfu) of Ad Flt1(1-3) do not exhibit lethality, and yet can still manifest responses such as increased hematocrit (FIG. 9A). Determination of whether the toxicity we have observed after injection of Ad Flt1 results from either excessive VEGF chelation by higher-affinity Flt1 [Waltenberger et al., J Biol Chem 269:26988-26995, 1994] or the distinct VEGF binding spectra of these receptors should aid the safety assessment of chronic VEGF-based anti-angiogenic therapies.

The present invention also provides a method for detecting efficacy of VEGF-inhibitor treatment. This comprises the steps of providing a first biological sample, preferably a blood sample, and measuring the hematocrit level in the sample before treatment with VEGF-inhibitor. The second sample is taken after treatment with VEGF-inhibitor. The sample may be taken at least 1 day after treatment with a VEGF-inhibitor. If the hematocrit level is increased in the second sample compared to the first sample, that indicates success in treatment with a VEGF-inhibitor.

Hemoglobin is the main element of red blood cells. It is a protein containing iron that allows the red blood cells to carry oxygen and waste products. Normal hemoglobin ranges between 14-18 grams per deciliter (g/dl) in men and 12-16 g/dl in women. A hemoglobin level of 10-14 g/dl in men and 10-12 g/dl in women is a sign of moderate anemia, while any number under 10 g/dl signals severe anemia in men and women.

Hematocrit measures the volume percentage of red blood cells in whole blood. Normal hematocrit levels range between 40-52% in men and 35-46% in women. Anemia is considered to be moderate when the hematocrit is between 35-40% in men and 30-35% in women and severe when the hematocrit falls below 35% in men and 30% in women.

Hematocrit measurement has been known and performed for decades having originally described by Wintrobe [J Clin Lab Med 15:287, 1929]. The level of hematocrit can be measured using a number of techniques well known to one skilled in the art. For example, the originally described method consisted of spinning anticoagulated whole blood in a specially designed tube, the Winthrop column, in a centrifuge until the red cells were packed to a constant volume. Length of the column of packed red cells was measured and total length of the blood column was measured and length of the packed red cell column was divided by the length of the total blood column, and the result expressed as percentage. [Id.] Method is sometimes referred to as the macrohematocrit method. The microhematocrit method utilizes a standardized glass or plastic capillary tube which is partially filled with anticoagulated blood. The end of the tube is sealed, and the tube is centrifuged in a high speed specifically designed centrifuge [Solomon et al. Transfusion 26:199-202, 1986]. Radioisotope dilution methods in which small amounts of a radioisotope labeled substance, such as albumin which will not enter the red cells, are added to whole blood. The relationship of numbers of counts in plasma to the counts in the total blood samples versus those in the plasma can be used to calculate the hematocrit. This is a very precise method (coefficient of variation {CV}=0.9%) [England et al. Br J Haematol 30:365-70, 1975]. Electrical impedance or conductivity of whole blood can be measured in both static samples and blood flowing through tubing, such as in pheresis instruments [de Vries et al. Med Biol Eng Comput 31:445-8, 1993]. A variety of instruments utilizing these techniques have been introduced, particularly for point of care testing [Cha et al. Physiol Meas 15:129-37, 1994]. Also optical methods and methods using weigh have been developed for determination of hematocrit [Steuer et al. Adv Ren Replace Ther 6:217-24, 1999; Joselow et al. Clin Chem 21:638-9, 1975].

All references cited in the above specification or in the Example below are herein incorporated by reference in their entirety.

The present invention is further illustrated by the following Example. The Example is provided to aid in the understanding of the invention and is not to be construed as a limitation thereof.

### EXAMPLE

Construction and Purification of Recombinant Adenoviruses

Murine Flk1-Fc cDNA was a gift of T. Niederman and contained the murine Flk1-Fc signal peptide, and the murine Flk1 ectodomain (to TIRRVRKEDGG [SEQ ID NO: 1], aa 731) fused to the murine IgG2α Fc fragment. Flk1-Fc cDNA was cloned with XbaI and BamHI ends into the adenoviral shuttle vector HIHG Add2 (J. Gray and R. C. M., unpublished), which contains a polylinker flanked by regions of homology from the E1 locus of adenovirus strain 5. Murine Flt1(1-3) was amplified by PCR from Flt-1 cDNA (S. Soker), facilitating addition of a C-terminal 6×His tag, digested with EcoRI and SalI and ligated into HIHG Add2. An alternative version of soluble Flt1 was produced by excising a DraIII-SalI fragment of HIHG Add2, containing the C-terminal 6×His tag, and ligating this into pDisplay (Invitrogen, Carlsbad, Calif.) at blunted BgIII and SalI sites to produce an in-frame fusion with the N-terminal HA tag in pDisplay. The resultant construct contained the Flt1(1-3) ectodomain with both HA and 6×His tags to facilitate ELISA detection, and was excised with EcoRI and SalI and ligated into EcoRI and SalI cut Add2. We have not observed functional differences between singly and doubly tagged soluble Flt1. A control unfused murine IgG2α cDNA (Lexigen Pharmaceuticals, Lexington, Mass.) was ligated into HIHG Add2 with XhoI and XbaI ends. Human sNRP cDNA with ABC domains and a C-terminal 6×His tag (S. Soker), was digested with BamHI and XbaI, the XbaI site blunted with T4 DNA polymerase, and cloned into BamHI/XhoI-digested Add2 in which the XhoI site had been blunted with T4.

Human angiostatin (AS) cDNA was amplified by PCR from human plasminogen cDNA, with amino acids 97-458 (lys 97-glu 458) comprising kringle domains 1-4 with an N-terminal hGH leader peptide. This PCR product was digested with BamHI and XhoI and cloned into the shuttle vector pAd-MDM (J. Gray and R. C. M., unpublished). The murine endostatin (ES) adenovirus donor plasmid was constructed by insertion of a murine ES coding sequence (HTHQD . . . TSFSK [SEQ ID NO: 2]) with collagen XVIII signal peptide (B. Olsen) into the shuttle vector pHIHG Add2 to generate pAdd2 mu endo II. An alternative murine ES donor plasmid, pAdd2 mu endo I, was constructed by PCR of murine collagen 18 cDNA (B. Olsen) to fuse the human growth hormone (hGH) signal sequence MATGSRTSLLLAFGLLCLPWLQEGSA [SEQ ID NO: 3] to the 184 amino acid murine ES coding sequence (HTHQD . . . TSFSK [SEQ ID NO: 2]) with flanking BamHI and XhoI sites. This PCR product was digested with BamHI and XhoI and cloned into the shuttle vector pAd-MDM. ES and AS inserts were sequenced on both strands to exclude PCR errors.

PacI-MfeI digests of shuttle vectors containing the transgene flanked by 2.0 kb and 1.4 kb of adenoviral sequences were recombined into the E1 locus of an E1 deleted Ad type 5 vector with a GM-CSF insert as described [Chartier et al., J Virol 70:4805-4810, 1996]. Positive adenoviral recombinants in which the transgene replaced GM-CSF were linearized with PacI and transfected into 293 cells, and agarose plaques were picked, expanded, and amplified. Virus was purified via CsCl gradient purification.

Protein Analysis of Virally Produced Endostatin and Flt1 (1-3)

C57B1/6 mice were injected with Ad mu endo II or Ad Flt1 (1-3) (10⁹ pfu by tail vein). After 3 days, mice were terminally bled and the respective proteins were purified from plasma using either heparin-sepharose chromatography with NaCl elution (ES) or Ni-agarose chromatography with imidizole elution (Flt1(1-3)). These purified proteins were transferred to PVDF membrane, and were digested in situ with trypsin, followed by N-terminal sequencing and mass spectroscopy.

ELISA Determination of Transgene Expression

Plasma samples were obtained by retroorbital puncture with heparinized capillary tubes after anaesthesia. Murine Flk1-Fc concentrations were determined by sandwich ELISA with anti-murine Flk1 primary (BD PharMingen, San Diego, Calif.) and anti-murine IgG2α Fc-HRP secondary (Jackson Immuno Research Laboratories, Inc., Bar Harbor, Me.). Murine Flt1 concentrations were determined by sandwich ELISA using antibodies against the N-terminal HA tag (Covance, Princeton, N.J.) and C-terminal His (Invitrogen, Carlsbad, Calif.) tag. Murine ES plasma levels were quantified by competition ELISA (Cytimmune Sciences, Inc., College Park, Md.) and human AS plasma levels by sandwich ELISA (EntreMed, Inc., Rockville, Md.).

Western Blot Determination of Transgene Expression

Plasma was analyzed by Western blot for Flk1-Fc (rat anti-murine Flk1, (BD PharMingen, San Diego, Calif.) or goat anti-murine Fc, (Jackson Immuno Research Laboratories, Inc., Bar Harbor, Me.), Flt1 (rabbit anti-His, Santa Cruz Biotechnology, Inc., Santa Cruz, Calif.), ES (rabbit anti-mouse ES, gift of K. Javaherian), AS (rabbit anti-human plasminogen, Axell, Accurate Chemical, Westbury, N.Y.) or sNRP (rabbit anti-His, Santa Cruz Biotechnology, Inc., Santa Cruz, Calif.). Development was performed with species-specific secondary Ab-HRP conjugates and chemoluminescence.

Tumor Cell Lines, Mice and Adenoviral Injections

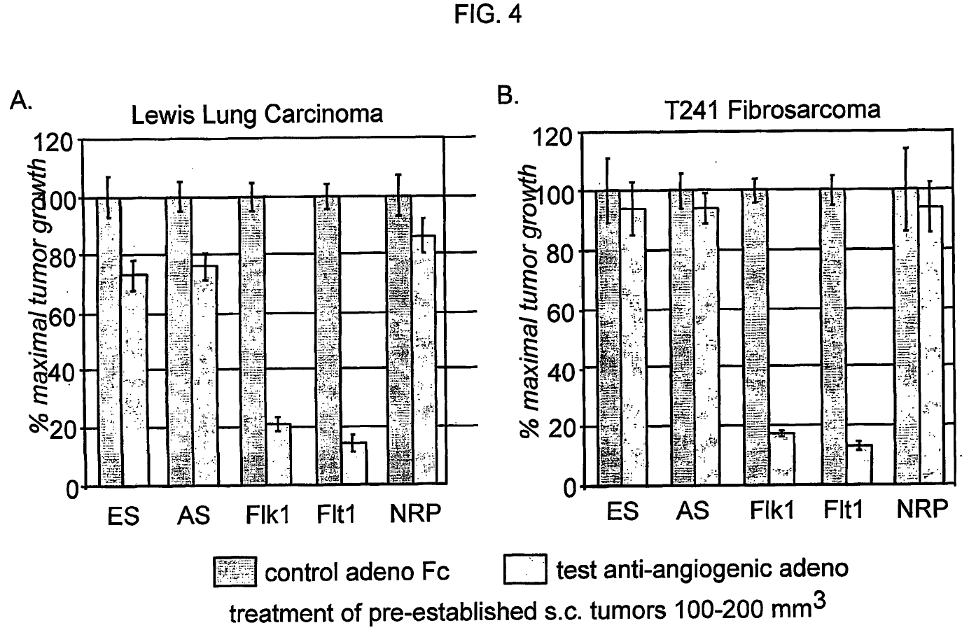

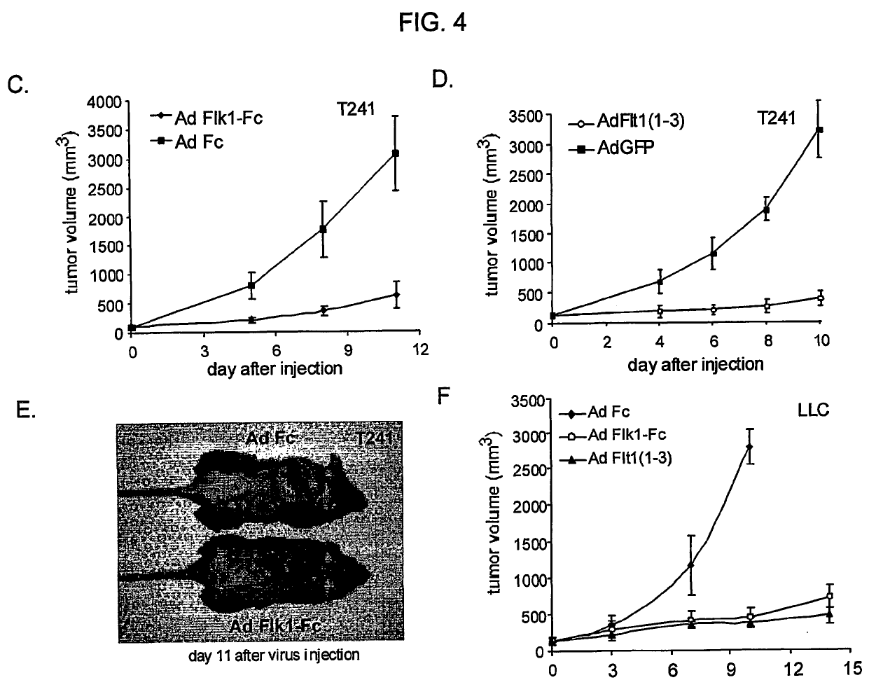

Murine LLC was passaged on the dorsal midline of C57B1/6 mice or in DMEM/10% FCS/PNS/L-glutamine. T241 murine fibrosarcoma was grown in DMEM/10% FCS/PNS/L-glutamine and human pancreatic BxPc3 adenocarcinoma in RPMI/10% FCS/PNS. Tumor cells (10⁶) were injected sub-cutaneously (s.c.) into the dorsal midline of C57B1/6 mice (8-10 weeks old) for murine tumors and SCID mice for human tumors, grown to 100-200 mm³ (typically 10-14 d) to demonstrate tumor take, and 10⁹ pfu of anti-angiogenic adenoviruses or the control adenovirus Ad Fc given by i.v. tail vein injection. In FIG. 4D, 7 Flt1 control animals received Ad GFP instead of Ad Fc, although we have not observed any differences in tumor inhibition with either control construct. Ad mu endo II was used in all endostatin experiments except in FIG. 4B, in which Ad mu endo I was used. Tumor size in mm³ was calculated by caliper measurements over a 10-14 day period using the formula 0.52×length (mm)×width² (mm), using width as the smaller dimension. P-values were determined using a 2-tailed t-test assuming unequal variances (Microsoft Excel).

Corneal Micropocket Assay

C57B1/6 mice received 10⁹ pfu i.v of anti-angiogenic adenoviruses or the control adenovirus Ad Fc two days before assay. Mice were anesthetized with avertin i.p. and the eye treated with topical proparacaine HCl (Ophthetic, Allergan, Inc., Irvine, Calif.). Hydron/sucralfate pellets containing VEGF-A₁₆₅ (R&D Systems, Minneapolis, Minn.) were implanted into a corneal micropocket at 1 mm from the limbus of both eyes under an operating microscope (Carl Zeiss, Inc., Thornwood, N.Y.) followed by intrastomal linear keratotomy using a microknife (Medtronic Xomed, Jacksonville, Fla.). A corneal micropocket was dissected towards the limbus with a von Graefe knife #3 (2×30 mm), followed by pellet implantation and application of topical erythromycin. After 5 days, neovascularization was quantitated using a slit lamp biomicroscope and the formula 2π×(VL/10)×(CH). P-values were determined using a 2-tailed t-test assuming unequal variances (Microsoft Excel).

Immunohistochemistry

Mice bearing LLC tumors on the dorsal midline of C57B1/6 mice at 50 mm³ received 10⁹ pfu i.v. of Ad Fc, Ad Flk1-Fc or Ad Flt1(1-3). After tumor growth to approximately 200 mm³, tumors were harvested, fixed in formalin, and parafin-embedded sections stained for CD31 using a biotin-strepavidin HRP system (Vectastain®, Vector Laboratories, Inc., Burlingame, Calif.). Microvessel areas were quantified by manual counting of hot-spots in sections.

Hematocrit Determination

Serum was collected from anaesthetized mice by retroorbital puncture and heparinized capillaries followed by centrifugation in a microcapillary centrifuge. Hematocrit was determined as the ratio of packed cell volume to total blood volume

Complete Blood Count Determination

Whole blood was collected from anaesthetized mice by retroorbital puncture using heparinized capillaries into EDTA coated microtainers. Flow cytometric analysis for determination of WBC, RBC and platelet number was performed according to standard procedures.

Determination of Reticulocyte Count

Whole blood from anaesthetized mice collected by retroorbital puncture using heparinized capillaries into EDTA coated microtainers was smeared onto microscope slides followed by methylene blue stain and manual counting of reticulocyte count as expressed as (%). Alternatively, flow cytometric analysis of reticulocyte count (automated reticulocyte count) from whole blood was performed.

FACS Analysis of Splenic and Bone Marrow Erythroid Precursors

Total spleen and bone marrow cells were extracted from mice post-sacrifice, passed through mesh filters to remove particulate matter, and resuspended in Iscove's Media supplemented with fetal calf serum. Subsequently, cells were incubated with anti-Ter119-phycoerythrin conjugated antibody (Pharmingen) and anti-CD45-FITC conjugated antibody (Pharmingen), followed by incubation with Hoechst 33342 dye. FACS analysis was performed by gating out the Hoechst negative population and the resultant % Ter119(+)CD45(−) cells calculated as a proportion of Hoechst positive cells.

Determination of Arterial Oxygen Concentration

Arterial catheters were placed into the left common carotid artery of anesthetized mice using the following technique, a personal communication from Dr. Drew Patterson, Stanford University: Prior to insertion of the catheter, each mouse underwent induction of general anesthesia using inhaled isoflurane. The animal's neck and a small portion of the animal's back were shaved using a small animal shear. After general anesthesia was achieved, a small midline neck incision was made. The left common carotid artery was isolated using a microscope. A suture was tied around the vessel approximately 0.25 cm below the skull base. Approximately 0.75 cm proximal to this point blood flow was disrupted using a vascular clamp. This provided a site in the vessel for catheter insertion. Proximal to the carotid artery suture, a small arterotomy incision was made using a curved 25 gauge needle. PE-10 tubing was then inserted into the vessel. The tubing was advanced 1.5 cm after the vascular clamp was released. The catheter was then secured with 5.0 suture. After back-bleeding the catheter into a syringe to insure no air bubbles resided in the tubing, the catheter was flushed with heparinized saline.

The catheter was then tunneled subcutaneously to the animal's back where it was allowed to exit through the skin into the shaved and prepped area. The catheter was then tied off and placed into a subcutaneous pouch for later retrieval and use. Twenty-four hours later, the catheter was retrieved and an ABG syringe inserted, followed by removal of 200 μl of whole blood, followed by automated determination of arterial pO₂, pH, pCO₂ and HCO₃ concentrations.

Construction and Characterization of Adenoviruses Encoding Soluble VEGF Receptors and Other Anti-angiogenic Gene Products

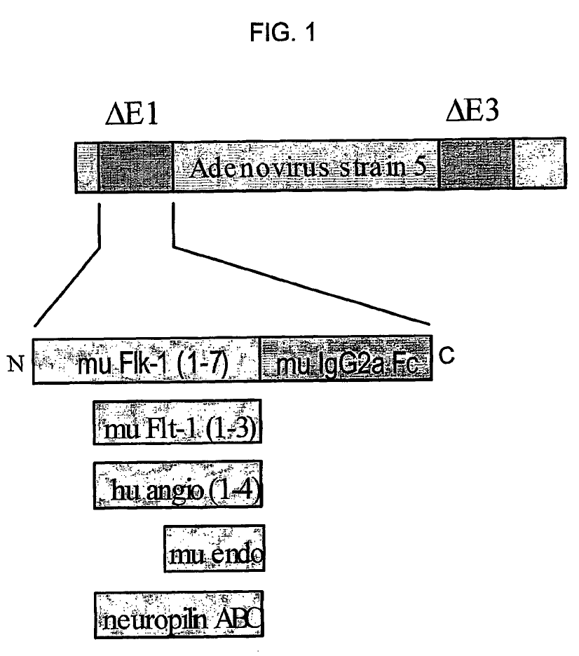

Using homologous recombination techniques in bacteria [Chartier et al., J Virol 70:4805-4810, 1996], DNA sequences encoding human angiostatin, murine endostatin, and the ligand-binding ectodomains of the VEGF receptors Flk1, Flt1 and neuropilin were introduced into the E1 region of a standard E1 deleted adenoviral vector (FIG. 1). Viruses encoding each of the gene products were generated after transfection of the different vector DNAs into 293 cells as previously described [Id.]. In the case of each vector, particle titers of approximately 10¹³/ml and infectious titers of approximately 10¹¹ plaque forming units/mil were routinely obtained, with a particle-to-infectivity ratios of 40-60.

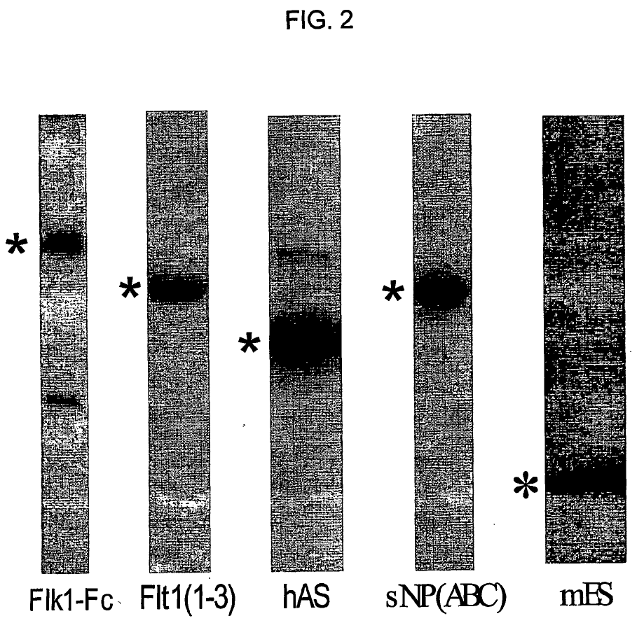

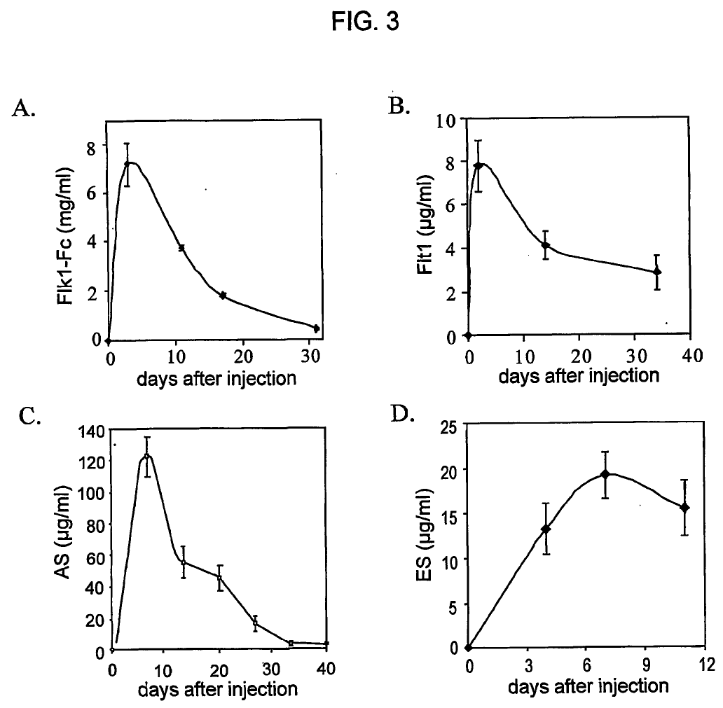

To evaluate the in vivo expression potential of the different viruses, 10⁹ plaque forming units of each virus was administered by intravenous or intramuscular routes into immunocompetent C57B1/6 mice. Transgene expression was easily detectable in the plasma of infected mice by Western blotting (FIG. 2). In the case of Flk-Fc, Flt1, angiostatin, and endostatin, plasma expression levels at different times post injection of virus were quantitated by sandwich ELISA (FIGS. 3A-D). Ad Flk1-Fc virus provided very high levels of protein expression (2-8 mg/ml) compared with Ad angiostatin (100-250 μg/ml), Ad endostatin (>10 μg/ml) and Flt1 (2-8 μg/ml), and the expression of all gene products declined progressively with time, consistent with the known transient nature of trangene expression afforded by first generation adenoviral vectors [Yang et al., Proc Natl Acad Sci USA 91:4407-4411, 1994]. In the case of animals injected with viruses encoding sNRP, Western blot analysis in conjunction with purified protein standards was used to estimate the peak serum concentration as >50 μg/ml (data not shown).

In vitro assays were used to confirm the functional activity of several of the adenovirus-expressed gene products. Vector encoded soluble Flt1 and Flk1-Fc proteins were both shown to inhibit VEGF-induced HUVEC proliferation in vitro, with IC₅₀'s of approximately 5 ng/ml and 100 ng/ml respectively (data not shown), paralleling reports the relative affinities of the two receptors for VEGF [Waltenberger et al., J Biol Chem 269:26988-26995, 1994]. In our experience, most of the functional assays previously described for endostatin and angiostatin (e.g., in vitro proliferation and migration assays) have been technically difficult to perform, and therefore were not utilized to confirm the functional activity of the two virus encoded gene products. Nevertheless, at least in the case of endostatin, we have shown that the virus encoded protein consistently inhibits endothelial migration in matrigel cultures in a manner similar to that observed with recombinant endostatin produced in yeast, baculovirus or myeloma cells (C. J. K., unpublished observations). In addition, mass spectroscopy and N-terminal sequencing analysis of virally encoded endostatin purified from the serum of mice injected with the corresponding virus indicated that the expected product was made (K. Javaherian and C. J. K., unpublished).

Systemic Inhibition of Tumor Growth by Soluble VEGF Receptors

The ability of each recombinant adenovirus vector to provide systemic inhibition of pre-established tumors was first evaluated in the aggressive Lewis lung carcinoma (LLC) model in which recombinant angiostatin and endostatin had been previously evaluated [O'Reilly et al., Cell 79:315-328, 1994; O'Reilly et al., Nat Med 2:689-692, 1996; O'Reilly et al., Cell 88:277-285, 1997; Boehm et al., Nature 390:404-407, 1997]. LLC cells were implanted subcutaneously on the dorsum of C57B1/6 mice for 10-14 days to a size of 100-150 mm³, consistent with definitive tumor engraftment, followed by i.v. injection of 10⁹ plaque forming units of the various adenoviruses. Under these conditions, adenoviral infection occurs primarily in liver without significant intratumoral infection (data not shown); consequently, any inhibition of tumor growth on the dorsum from protein produced in a remote site (i.e. liver) would presumably occur by a systemic mechanism.

In mice bearing pre-existing LLC tumors, i.v. injection of Ad Fc resulted in rapid tumor growth often requiring sacrifice by day 14-post virus injection (FIG. 4A). No significant difference was observed between tumor growth in Ad Fc- and PBS-treated animals (unpublished observations). In contrast, after 10-14 days of treatment, tumors in either Ad Flk1-Fc- or Ad Flt1-injected mice exhibited approximately 80% growth inhibition relative to controls, which was statistically significant compared with the Ad Fc control virus (p<0.000001). In contrast, LLC growth was less strongly inhibited by Ad endostatin (27%, p=0.004), Ad angiostatin (24%, p=0.001) or Ad neuropilin (14%, p=0.15) (FIG. 4A). The anti-tumor effects of both Ad Flk1-Fc and Ad Flt1 were dose dependent, with the minimal day 3 plasma concentrations for effective systemic tumor suppression being approximately >1 mg/ml for Flk1-Fc and >2 μg/ml for Flt1(1-3) (F. Farnebo, E. Yu., B. Swearingen, and C. K., unpublished). In most cases, tumor growth eventually supervened after 3-4 weeks (data not shown). Although the studies do not rule out acquired endothelial and/or tumor resistance as the mechanism underlying the observed escape from inhibition, the rapid decline of vector-mediated gene expression over time most likely accounts for the observed results.

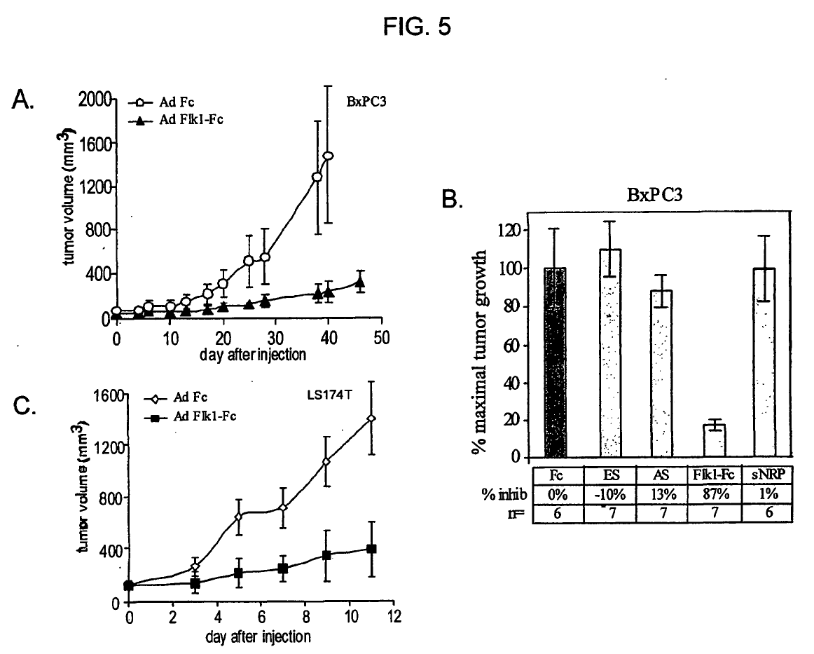

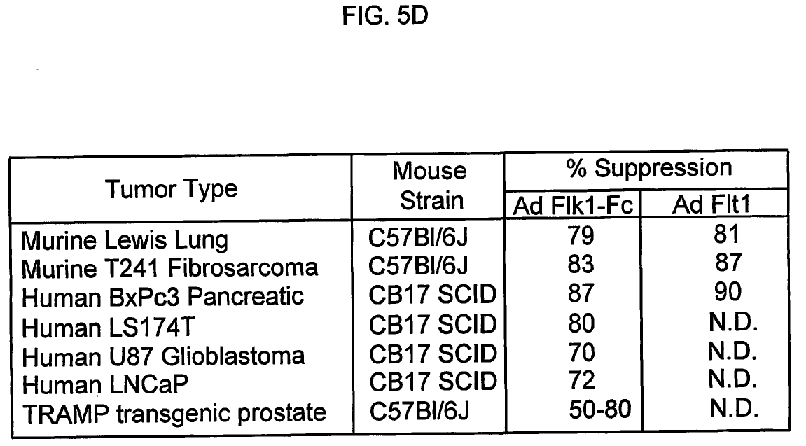

Superior anti-tumor efficacy for soluble VEGF receptors over angiostatin or endostatin was similarly observed in a syngeneic murine T241 fibrosarcoma-C57B1/6 tumor model (FIG. 4B) and in a xenogeneic BxPc3-SCID tumor model (FIGS. 5A, 5B). In the case of the T241 model, strong tumor suppression was again exhibited by Ad Flk1-Fc (83%, p<0.000001) and Ad Flt1 (87%, p<0.000001); yet, in this model, little or no inhibition of tumor growth was achieved by Ad endostatin (6%, p=0.71), Ad angiostatin (6%, p=0.86) or Ad neuropilin (6%, p=0.77) (FIGS. 4B-D). In the case of the BxPc3 model, Ad Flk1-Fc produced a strong suppression of tumor growth (83%, p=0.025), while Ad endostatin, Ad sNRP or Ad angiostatin did not significantly inhibit growth of preestablished BxPC3 tumors with <12% inhibition (p=0.60-0.98) (FIGS. 5A-B). In a last series of experiments, Ad Flk-Fc was also shown to strongly inhibit tumor growth in another xenogenic tumor model involving LS174T human colon carcinoma and SCID mice (79%, p=0.0003) (FIG. 5C).

Overall, either of the soluble VEGF receptors Flk1-Fc or Flt1 exhibit potent and broad-spectrum suppression of human and murine tumors in subcutaneous, orthotopic and transgenic models (summarized in FIG. 5D). In addition to the tumor types described above, we have also observed strong activity of Flk1-Fc against orthotopically implanted human LNCaP prostate carcinoma in SCID mice (C. J. K, R Christofferson, F. Farnebo and R. C. M., unpublished), against orthotopically implanted human U87 glioblastoma in SCID mice (R. Carter, C. J. K. and R. C. M., unpublished), and against TRAMP transgenic prostate carcinoma in C57B1/6 mice (C. Becker, C. J. K. and B. Zetter, unpublished). These data reinforce the systemic anti-tumor efficacy of these soluble VEGF receptors.

Systemic Inhibition of Tumor Angiogenesis by Soluble VEGF Receptors

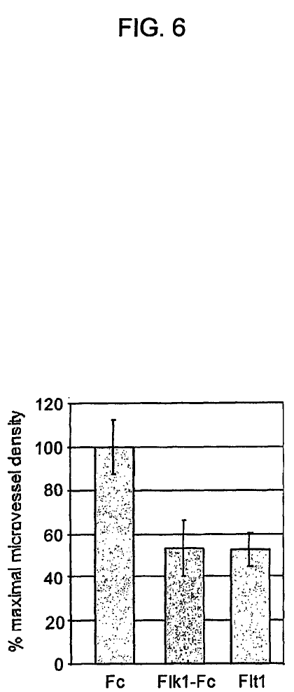

Microvessel density has been extensively used as a marker for tumor angiogenesis, tumor grade, and inhibition of microvessel density as a measure of anti-angiogenic activity [Weidner, Am J Pathol 147:9-19, 1995]. To evaluate the mechanism for Ad Flk1-Fc and Ad Flt1 suppression of tumor growth, the microvessel density of treated versus non-treated tumors was measured. Lewis lung carcinoma cells (LLC, 10⁶ cells) were implanted subcutaneously in the dorsal midline of C57B1/6 mice, and tumors were allowed to grow to approximately 50 mm³. The tumor-bearing mice then received i.v. injections of either Ad Flk1-Fc, Ad Flt1 or Ad Fc, followed by confirmation of expression levels by ELISA, and sacrifice for histologic analysis after reaching a size of 200 mm³. Immunohistochemistry for the endothelial antigen CD31 demonstrated an approximately 50% reduction of microvessel density in Flt1 and Flk1-Fc mice relative to Fc mice (FIG. 6). Parallel administration of Ad lac Z virus produced strong staining in liver and minor staining in lung, but did not produce significant intratumoral lac Z staining (data not shown).

Systemic Inhibition of VEGF-stimulated Corneal Angiogenesis by Anti-angiogenic Adenoviruses

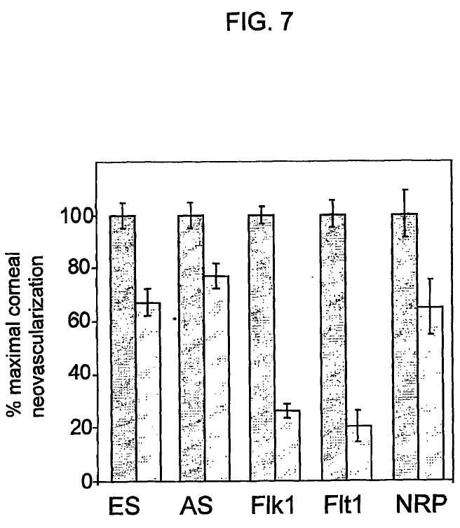

The ability of the different adenovirus-produced proteins to provide systemic inhibition of angiogenesis in vivo was also evaluated in a VEGF-dependent corneal neovascularization model. C57B1/6 mice received i.v. injections of the various adenoviruses followed after 2 days by implantation of hydron pellets containing human VEGF-A₁₆₅ into the mouse cornea. Plasma expression of the appropriate transgene was confirmed by ELISA or Western blotting, followed by quantitation of corneal neovascularization 5 days after pellet implantation. In mice receiving VEGF pellets, corneal neovascularization was strongly inhibited by Ad Flk1-Fc (74%, p<0.0000001) or Ad Flt1 (80%, p<0.0000001), which was statistically significant relative to the Ad Fc control virus (FIG. 7). VEGF-stimulated corneal angiogenesis was inhibited to a lesser degree by Ad endostatin (33%, p=0.0001), Ad angiostatin (23%, p=0.002) or Ad neuropilin (35%, p=0.027) (FIG. 7). These data confirm the relative rank order of anti-tumor efficacy noted with several tumor models (FIG. 4 and 5), and support an anti-angiogenic mechanism as suggested by decreased microvessel density.

Soluble VEGF Receptor Treatment Produces Elevated Hematocrit

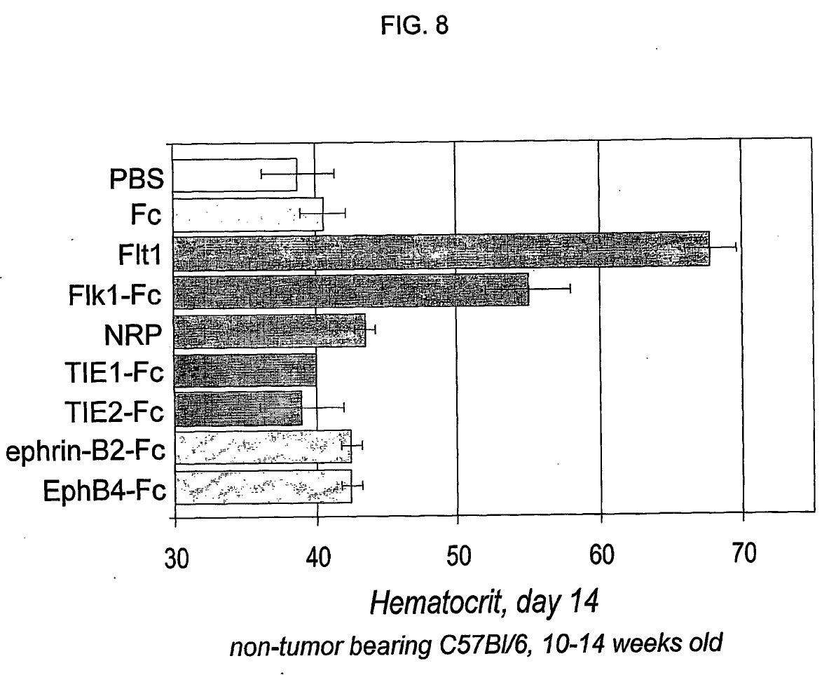

Surprisingly, non-tumor-bearing adult mice treated with soluble Flk1 or Flt1 adenoviruses (Ad Flk1-Fc, Ad Flt1) exhibited hematocrits in the 55-70% range after 14 days, as opposed to hematocrit of approximately 40% in untreated mice (FIG. 8). Notably, elevation of hematocrit was not observed in mice receiving adenoviruses encoding soluble ectodomains of the endothelial receptor tyrosine kinases TIE1, TIE2 and ephB4, or of the endothelial receptor ephrin-B2 and NRP1 (FIG. 8). Similarly increased hematocrit was not observed after injection of PBS or Ad Fc (FIG. 8), Although NRP1 functions as a VEGF receptor, the association of NRP1 with the C-terminus of VEGF is not predicted to interrupt VEGF receptor tyrosine kinase signalling via Flk1 or Flt1, which associate with more N-terminal domains of VEGF. The lack of stimulation of hematocrit observed with NRP (FIG. 8) parallels lack of anti-tumor activity (FIG. 4), and suggests that inhibition of VEGFR tyrosine kinase signalling may be relevant for eliciting both anti-angiogenic and hematopoietic effects.

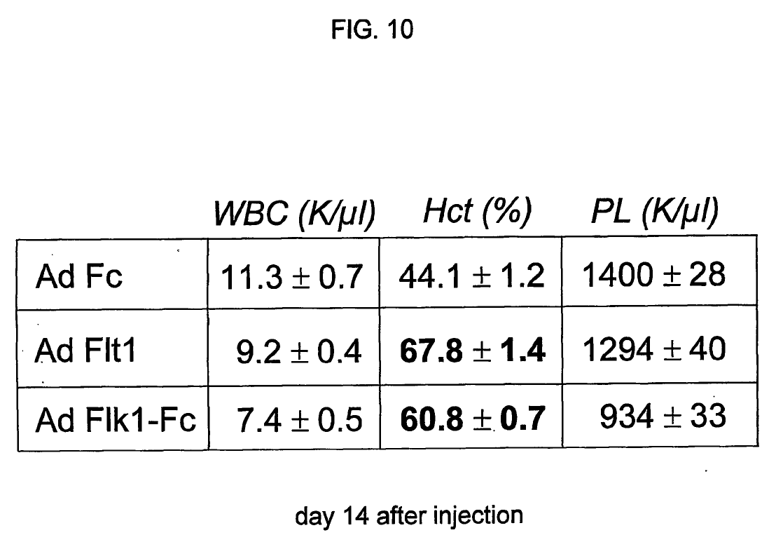

The polycythemia observed with both Flt1 and Flk1 -Fc treatment exhibited dose- and time-dependence, with progressive elevations in hematocrit observed over a 21-28 day period (FIGS. 9A-B). Hematocrit levels following Flt1 treatment (65-75) were consistently higher than following Flk1-Fc treatment (55-60) despite approximately 400-fold differences in expression between Flk1-Fc (3200 μg/ml) and Flt1 (8 μg/ml). These differences in relative efficacy parallel anti-tumor activity (FIGS. 1 and 4) and are consistent with the established higher affinity of Flt1 than Flk1 for VEGF. We have observed significant and sustained elevations in hematocrit following day 3 (peak) plasma levels of as little as 75-300 ng/ml for Flt1, indicating the potency of this particular soluble VEGF receptor (FIG. 9A). Notably, amongst the major hematopoietic lineages, increases were only noted in RBC, while slight decreases were noted in WBC and platelets, although these decreases were not of clinical significance (FIG. 10). In total, these data indicate a previously unsuspected role for VEGF in maintenance of basal hematocrit levels, with inhibition of VEGF function resulting in polycythemia.

Polycythemia in Mice Treated with Soluble VEGF Receptors does not Result from Hypoxia or Dehydration

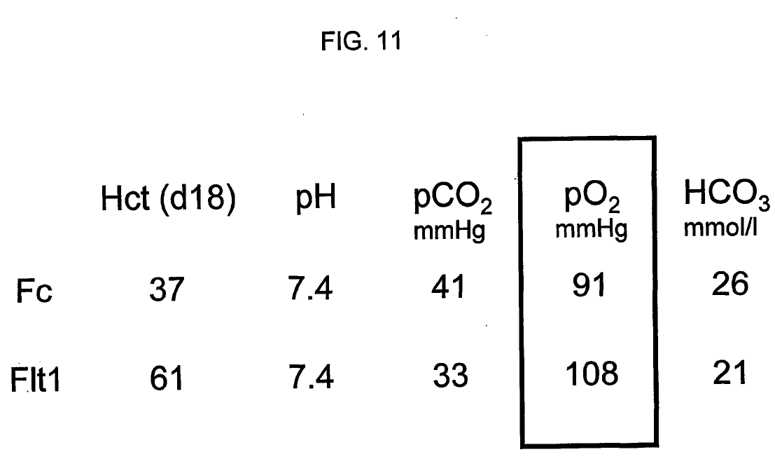

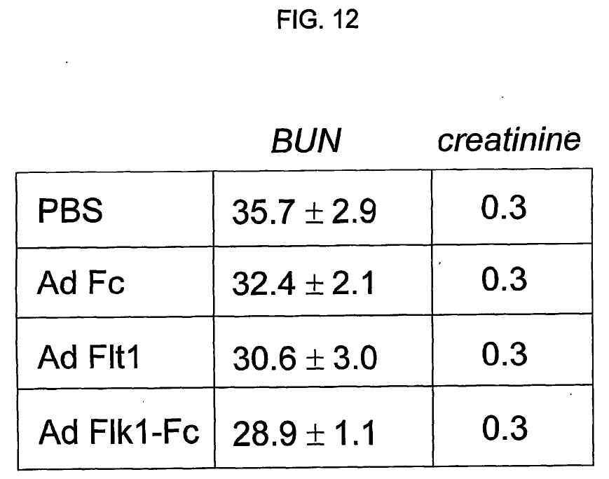

Elevations in hematocrit, or polycythemia, can be observed for trivial reasons, such as dehydration. True polycythemia (absolute erythrocytosis) can be further divided into primary (such as in polycythemia vera) or secondary (such as in response to hypoxemia). To rule out systemic hypoxemia, arterial blood gas measurements were performed on resting mice 14 days after i.v. administration of Ad Fc or Ad Flt1. These measurements revealed similar and normal pO₂ values in both animals despite increased hematocrit in the Flt1-treated mouse (FIG. 11). To rule out dehydation, blood urea nitrogen (BUN)/creatinine (Cr) ratios were measured 14 days after Ad Flk1-Fc or Ad Flt1 treatment. Under these conditions, despite elevated hematocrit in Flt1 and Flk1-Fc animals, BUN/Cr ratios were unaltered relative to control PBS or Fc animals, suggesting normal intravascular volume status (FIG. 12). Further supporting intravascular volume status and lack of clinical dehydration, normal skin turgor was observed, and animals did not exhibit weight loss during intervals of progressive polycythemia (data not shown). These observations rule out hypoxia and dehydration as trivial etiologies of the polycythemia observed after soluble VEGF receptor treatment.

Elevated Hematocrit in Soluble VEGFR Treated Mice is Accompanied by Polychromasia and Reticulocytosis

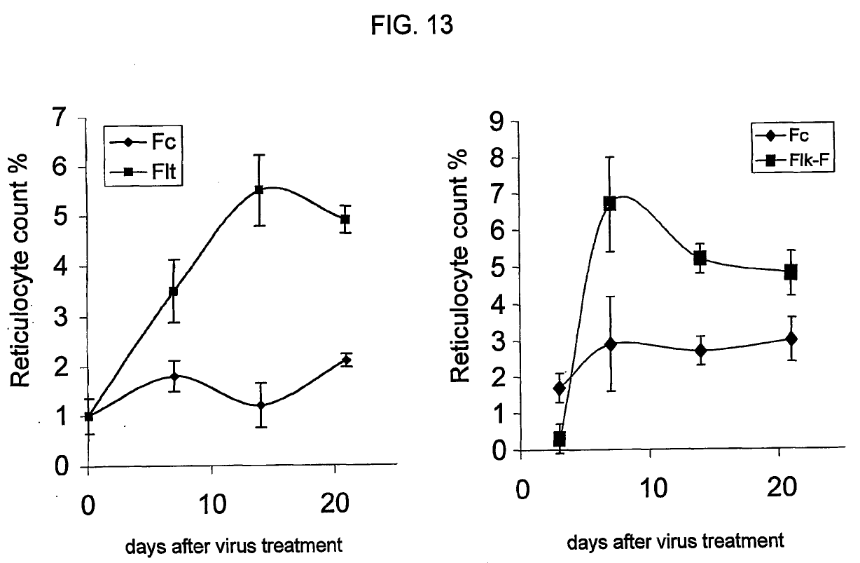

Several independent methods were utilized to formally demonstrate stimulate red blood cell production following soluble VEGFR treatment. Examination of the blood smear from animals treated with Ad Flt1 and Ad Flk1-Fc demonstrated pronounced polychromasia relative to Ad Fc-treated animals. During RBC production states, erythrocytes are released from the bone marrow before loss of residual RNA in effort to meet production demand. These RNA-containing “reticulocytes” can be identified by methylene blue staining, with positive cells typically corresponding to polychromatic cells, with increases in reticulocyte count (% reticulocytes compared with total RBC) used as a conventionally accepted measure of enhanced erythrocytosis. Methylene blue staining of peripheral blood smears from Ad Flt1- and Ad Flk1-Fc-treated mice revealed readily detectable reticulocytes compared with Ad Fc animals with 2-4 fold enhancement in the reticulocyte count (FIG. 13), consistent with increased RBC production following VEGF blockade with soluble receptors.

Soluble VEGF Receptor Treatment Stimulates Production of Ter119(+) CD45(−) Erythroid Precursors in Bone Marrow and Spleen

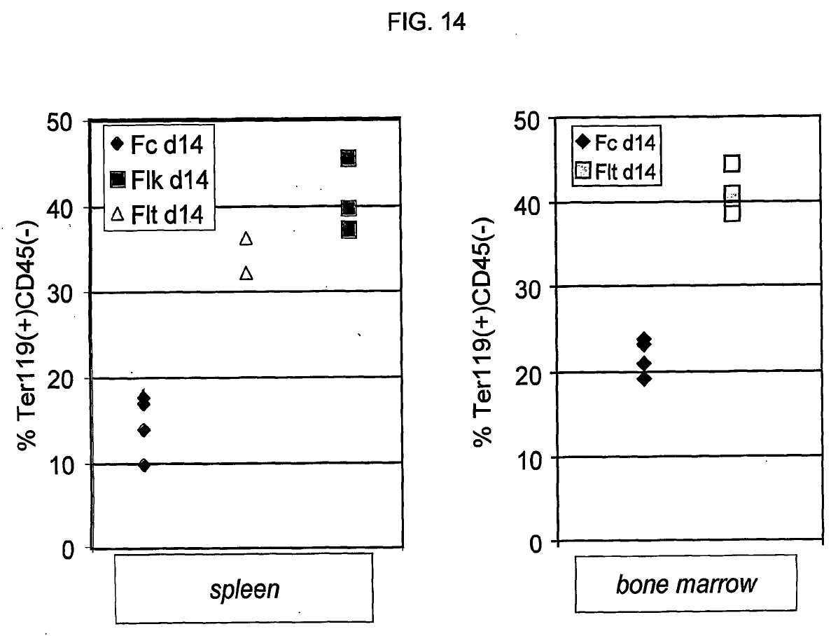

To further confirm increased RBC production following soluble VEGFR treatment, erythroid precursors in bone marrow and spleen were quantitated using FACS. Bone marrow or spleen cells which are negative for the pan-lymphocyte marker CD45, but positive for the erythroid precursor antigen Ter119 represent an early erythroid progenitor population which undergoes induction during RBC production states. FACS analysis of bone marrow and spleen from animals treated with Ad Flk1-Fc or Ad Flt1 revealed strong induction of Ter119(+) CD45(−) erythroid precursors relative to control Ad Fc animals (FIGS. 14A-B), again demonstrating increased RBC production following VEGF blockade with soluble receptors.

Although the foregoing invention has been described in some detail by way of illustration and an example for purposes of clarity of understanding, it will be readily apparent to those of ordinary skill in the art in light of the teachings of this invention that certain changes and modifications may be made thereto without departing from the spirit or scope of the appended claims.

We claim:

1. A method of treating a subject having disease or disorder associated with VEGF, the method comprising administering to the subject an angiogenesis inhibiting amount of a pharmaceutical composition comprising a truncated, soluble Flk1/KDR receptor and a pharmaceutically acceptable carrier or diluent.

2. The method of claim 1, wherein administering comprises a viral vector containing a nucleic acid encoding the truncated, soluble Flk1/KDR receptor.

3. The method of claim 2, wherein the viral vector is an adenovirus.

4. The method of claim 2, wherein the viral vector is a gutless adenovirus.

5. The method of claim 1, wherein the truncated, soluble Flk1/KDR receptor is a recombinant protein.

6. The method of claim 5, wherein the recombinant protein is part of a fusion protein.

7. The method of claim 1, wherein the disease or disorder associated with VEGF a metastatic tumor or inappropriate angiogenesis including retinal neovascularization, tumor growth, hemangioma, a solid tumor, leukemia, metastatic tumor, psoriasis, neovascular glaucoma, diabetic retinopathy, arthritis, endometriosis, and retinopathy of prematurity.

8. The method of claim 1, wherein the subject is a mammal.

9. The method of claim 1, wherein the subject is a human.

10. The method of claim 1, wherein the disease or disorder is a solid tumor.

11. The method of claim 1, wherein the subject has previously been treated with one or more conventional cancer treatment method including chemotherapy and radiation therapy and wherein the subject is suffering from decreased hematocrit level.

12. A method of increasing hematocrit level in a subject suffering from a decreased hematocrit level comprising administering to the individual an efficient amount of a pharmaceutical composition comprising an angiogenesis inhibitor.

13. The method of claim 12, wherein the angiogenesis inhibitor is encoded by a nucleic acid sequence contained within a vector.

14. The method of claim 13, wherein the vector is a viral vector.

15. The method of claim 14, wherein the viral vector is a gutless adenovirus vector.

16. The method of claim 12, wherein the angiogenesis inhibitor is a VEGF inhibitor.

17. The method of claim 16, wherein the VEGF inhibitor is selected from the group consisting of soluble fragment of Flt-1, Flt-4, neuropilin-1, neuropilin-2, and Flk/KDR.

18. The method of claim 12, wherein the angiogenesis inhibitor is a truncated, soluble Flk1/KDR.

19. The method of claim 12, wherein the individual has previously been treated with chemotherapy or radiation therapy.

20. The method of claim 12, wherein the angiogenesis inhibitor is a mixture of two or more angiogenesis inhibitors.

21. A method of treating a subject with a low hematocrit level and a disease or disorder associated with inappropriate angiogenesis comprising administering to said subject an effective amount of an angiogenesis inhibitor.

22. The method of claim 21 wherein the angiogenesis inhibitor is a truncated, soluble Flk/KDR.

23. A method of measuring the efficacy of VEGF-inhibitor treatment comprising the steps of:
a) providing a first biological sample of a subject and measuring a hematocrit level in the first biological sample;
b) administering a VEGF-inhibitor to the subject; and
c) providing a second biological sample of a subject and measuring the hematocrit level in the second biological sample, wherein an increased hematocrit level in the second biological sample compared to the hematocrit level in the first biological sample indicates that the VEGF-inhibitor treatment of the subject has been effective.

24. A method of treating a preexisting tumor comprising administering to the subject a tumor growth inhibiting amount of a pharmaceutical composition comprising a truncated, soluble Flt1 receptor and a pharmaceutically acceptable carrier or diluent.
